(19) United States
(12) Patent Application Publication
(10) Pub. No.: US 2026/0144915 A1
(43) Pub. Date: May 28, 2026

(54) Biomimetic Vascularized 3D Reinforced PCL/Collagen/In-Situ HAPclay Scaffolds With Iliotibial Band Cavity for Anterior Cruciate Ligament (ACL) Regeneration

(71) Applicants: Sanford Health, Sioux Falls, SD (US); NDSU Research Foundation, Fargo, ND (US)

(72) Inventors: Dinesh Ramanath Katti, Fargo, ND (US); Kalpana Shastri Katti, Fargo, ND (US); Pooyan Vahidi Pashaki, Fargo, ND (US); Benjamin Carter Noonan, West Fargo, ND (US)

(21) Appl. No.: 19/402,004

(22) Filed: Nov. 26, 2025

Related U.S. Application Data
(60) Provisional application No. 63/725,701, filed on Nov. 27, 2024.

(51) Int. Cl. A61L 27/36; A61L 27/12; A61L 27/18; A61L 27/24; A61L 27/38; A61L 27/54; A61L 27/58

(52) U.S. Cl. CPC A61L 27/3662; A61L 27/12; A61L 27/18; A61L 27/24; A61L 27/3834; A61L 27/54; A61L 27/58

(57) ABSTRACT

The disclosure relates to compositions useful for ligament and tendon repair and methods of preparing the same. The disclosure is particularly suitable for repair of ligament ruptures, for example, anterior cruciate ligament ruptures. In an aspect of the invention, the compositions comprise a biocompatible clay, a polymer, a polypeptide, a calcium-based mineral that form a scaffold, as well as a bioabsorbable reinforcement structure incorporated thereon and/or therein. Methods of use and manufacture of the compositions, as well as kits including the compositions, are also provided.

### CROSS REFERENCE TO RELATED APPLICATIONS

This application claims the benefit of U.S. Provisional Patent Application No. 63/725,701, filed Nov. 27, 2024, which is incorporated by reference herein in its entirety.

### FIELD OF THE DISCLOSURE

The disclosure relates to compositions that include a composite material and bioabsorbable reinforcement structure, which form a scaffold useful for ligament and tendon repair. Scaffolds of the disclosure are particularly suitable for repair of a ruptured anterior cruciate ligament. The disclosure further relates to methods of preparing such compositions.

### BACKGROUND

The anterior cruciate ligament (ACL) is a crucial structural component in the knee joint. Excessive loads while performing different physical activities can lead to various degrees of ligament injury, including tears or complete rupture. The primary goal of ACL reconstruction is to provide functional stability to the knee joint. Current ACL reconstruction strategies include autograft (from the patient's body), allograft (from a donor), as well as tissue engineering approaches to replace the native ligament.

Challenges associated with conventional surgical methods encouraged tissue engineering approaches for mimicking natural ligaments. However, despite numerous attempts to generate tissue-engineered ACL have been developed, only a small fraction of ACL repair scaffolds has been used in clinical settings. The lack of success is understandable given the need for a procedure-relevant biodegradation rate and a high absorption rate. These scaffolds must absorb blood efficiently, potentially enriched with growth factors, to accelerate ligament formation. Given these requirements, the repair scaffolds should improve the biological responses and also have sufficient mechanical properties (e.g., adequate durability to survive the surgical procedure).

Some natural biomaterials, such as collagen and gelatin, possess desirable characteristics (e.g., can form 3D structures with high porosity, enabling rapid fluid absorption). However, they also suffer from rapid disintegration in relevant conditions (e.g., in synovial fluid in the knee or shoulder), before adequate ligament formation occurs.

Thus, there remains a critical, unmet need for scaffolds that can withstand physiologically relevant synovial environments, provide durable mechanical and biological support, absorb fluids efficiently, and enable customized, surgeon-friendly application for all patient types. The present invention satisfies this need by introducing a new class of composite scaffolds to achieve superior outcomes in ACL and soft tissue repair.

### SUMMARY

The disclosure relates to compositions that include a composite material and bioabsorbable reinforcement structure, which together form a scaffold useful for ligament and tendon repair. The disclosure further relates to methods of preparing such compositions and surgical methods utilizing such compositions, and kits related thereto. Advantages of scaffolds of the disclosure include having many properties that are desirable for ligament and tendon repair, and particularly ligament repair in the knee, including, for example, mechanical robustness necessary for arthroscopic surgical use, absorptive properties, controlled degradation properties, and desirable pore size.

In an aspect, a composition is provided that includes a composite and a bioabsorbable reinforcement structure. The composite includes a biocompatible clay, a polymer, a polypeptide, and a calcium-based mineral and forms a three-dimensional scaffold with a hole formed therethrough along an axis of the scaffold. The bioabsorbable reinforcement structure is located on an outer surface of the scaffold, an inner surface of the scaffold, within the scaffold, or combinations thereof.

In some embodiments of this aspect, the biocompatible clay comprises smectite minerals—such as bentonite, beidellite, hectorite, montmorillonite, and the like—including sodium or calcium salt forms, which may be modified with amino acids, for example, 5-aminovaleric acid. The calcium-based mineral may be hydroxyapatite, mineralized in situ within the clay. The polymer can include polycaprolactone (PCL), polylactide (PLA), polyglycolide (PGA), PLGA, chitosan, as well as blends thereof. The polypeptide may be natural, recombinant, or modified collagen, collagen mimetic, or gelatin.

Further embodiments provide for the adjustment of component ratios, e.g., 40-70% collagen, 5-12% in situ HAPclay, and remainder polymer, to tune scaffold properties including degradation rate, fluid absorption, porosity, strength, and bioactivity. The reinforcement structure may be absorbable suture material, arranged in parallel, spiral, zig-zag, or composite patterns, and manufactured from natural or synthetic polymers (e.g., PDS, Vicryl, Monocryl, gut, or blends) as single or mesh filaments. Embodiments encompass a range of scaffold geometries and internal diameters, surface textures or porosities, as well as inclusion of additional agents-such as stem cells, growth factors, drugs, or gene constructs—by impregnation, coating, or mixing. The scaffold may exhibit tailored mechanical and biological properties, including strength, blood absorption up to 85% or more, and promotion of tenogenic and angiogenic gene expression.

In some embodiments, the composition further comprises one or more additional agent, including, for example, an amino acid, anesthetic, antibiotic, antiangiogenic agent, antibody, anticoagulant, biomaterial, bone morphogenetic proteins, carbohydrate, cell, drug, therapeutic agents, plant extracts, electrolyte, growth factor, vascular endothelial growth factor, immunomodulator, inorganic material, lipid, mineral, oligonucleotide, osteoblast, osteoclast, osteo stem cell, peptide, progenitor, protein, therapeutic agent, tissue, tissue or cell aggregate, vasoactive agent, and combinations thereof.

In another aspect, a method for preparing compositions of the disclosure is provided, including dissolving a biocompatible clay in an aqueous solvent to form a dissolved clay; adding a calcium-based mineral to the dissolved clay to form a mineralized clay precipitate; optionally modifying the clay with amino acids; separating, drying and grinding the mineralized clay precipitate to form a mineralized clay powder; dissolving the polymer in a solvent to form a dissolved polymer; adding a polypeptide, e.g., collagen, gelatin, to the dissolved polymer to form a polymer and polypeptide mixture; adding the mineralized clay powder to the polymer and polypeptide mixture to form a clay, polymer, and polypeptide mixture; adding a bioabsorbable reinforcement structure within a mold of a desired three dimensional shape; adding the clay, polymer, and polypeptide mixture to the mold; placing an occluding device (e.g., a stem or tube) through the clay, polymer, and polypeptide mixture to form a hole in the composite once the mixture is in solid form and the occluding device or stem is removed; freeze extracting the clay, polymer, and polypeptide mixture; forming a scaffold in the desired three dimensional shape with a hole therethrough and a bioabsorbable reinforcement structure, once removed from the mold and removing the stem.

In another aspect, the disclosure provides a method of using the disclosed composition in surgical procedures, e.g., repairing a tear or rupture of a ligament or tendon. In some embodiments, the method comprises harvesting an autograft or allograft (such as an iliotibial band), sliding or threading the scaffold over the graft, and anchoring the graft at two or more anatomic points for ACL repair or tendon reconstruction. Further embodiments include procedures compatible with minimally invasive or extra-articular approaches, such as “over-the-top” or Lemaire techniques, as well as using the scaffold for osteointegration, tissue regeneration, or delivering biological agents intraoperatively.

In another aspect, the disclosure provides a kit including one or more scaffolds as described, optionally packaged with surgical or preparation accessories (e.g., tools, suture material, injectable agents) and instructions for clinical use. Embodiments may include pre-loaded cell or growth factor formulations, delivery devices, or other modular components for surgical workflow enhancement.

In an aspect, the disclosure provides compositions with specified component ratios and process features. Embodiments include specific parameter values, mechanical attributes, absorption and retention characteristics, biological activity metrics, or use-case scenarios, covering optimized scaffold variants and their experimentally validated properties.

While multiple embodiments are disclosed, still other embodiments of the present disclosure will become apparent to those skilled in the art from the following detailed description, which shows and describes illustrative embodiments of the disclosure. Accordingly, the figures and detailed description are to be regarded as illustrative in nature and not restrictive.

### BRIEF DESCRIPTION OF THE DRAWINGS

FIG. 1A shows a 3D knee joint model with a tunnel in the tibia and the femur. FIG. 1B shows a schematic of an injured ACL, replaced by IT Band, and scaffold. FIG. 1C shows the construct including scaffold, sutures, internal brace, and IT-Band passing through the cannula (FIG. 1C).

FIG. 2A to FIG. 2D show PBS absorption of the scaffolds (after 2 mins, 5 mins, 1 hour, and 24 hours). CO40HC5, CO40HC7, CO40HC10, CO40HC12 are shown in FIG. 2A. CO50HC5, CO50HC7, CO50HC10, CO50HC12 are shown in FIG. 2B. CO60HC5, CO60HC7, CO60HC10, CO60HC12 are shown in FIG. 2C. CO70HC5, CO40HC7, CO40HC10, CO40HC12 are shown in FIG. 2D. A one-way ANOVA was conducted, followed by Tukey's post hoc analysis. Statistical significance was denoted as p<0.05, p<0.01, and p<0.001 (n=5). FIG. 2E shows a repair scaffold schematic representation. FIG. 2F shows placing the CO60HC10 scaffold in the knee joint around the injured ACL, and injecting patient blood. FIG. 2G shows PBS absorption of the PCL scaffolds after 2 mins, 5 mins, 15 mins, 1 hour, and 24 hours.

FIG. 3A shows simulated synovial fluid constituents for degradation study. FIG. 3B shows the schematic illustrates the placement of scaffolds in a tube for degeneration studies. FIG. 3C shows degradation percentage (%) of CO40HC10, CO50HC10, CO60HC10, CO70HC10 scaffolds (1, 8, 15, 22, and 29 days) n=5; mean±SD. FIG. 3D shows Young modulus values: representative stress-strain curves obtained for CO40HC5, CO40HC7, CO40HC10, CO40HC12, CO50HC5, CO50HC7, CO50HC10, CO50HC12, CO60HC5, CO60HC7, CO60HC10, CO70HC12, CO70HC5, CO40HC7, CO40HC10, CO40HC12 scaffolds. FIG. 3E shows representative compressive elastic moduli of 40% collagen scaffolds with changing concentration of in situ HAPclay including 5% in situ HAPclay, 7% in situ HAPclay, 10% in situ HAPclay, 12% in situ HAPclay. FIG. 3F shows representative compressive elastic moduli of 50% collagen scaffolds with changing the concentration of in situ HAPclay including: 5% in situ HAPclay, 7% in situ HAPclay, 10% in situ HAPclay, 12% in situ HAPclay. FIG. 3G shows representative compressive elastic moduli of 60% collagen scaffolds with varying percentages of in situ HAPclay, including 5% in situ HAPclay, 7% in situ HAPclay, 10% in situ HAPclay, 12% in situ HAPclay. FIG. 3H shows representative compressive elastic moduli of 70% collagen scaffolds with varying different percentages of in situ HAPclay including 5% in situ HAPclay, 7% in situ HAPclay, 10% in situ HAPclay, 12% in situ HAPclay. A one-way ANOVA was conducted, followed by Tukey's post hoc analysis. Statistical significance was denoted as p<0.05, p<0.01, and p<0.001 (n=5). FIG. 3I to FIG. 3K show degradation percentage (%) of scaffolds (1, 8, 15, 22, and 29 days). n=5; mean±SD. CO40HC5, CO50HC5, CO60HC5, and CO70HC5 are shown in FIG. 3I. O40HC7, CO50HC7, CO60HC7, and CO70HC7 are shown in FIG. 3J. CO40HC12, CO50HC12, CO60HC12, and CO70HC12 are shown in FIG. 3K. FIGS. 3I, 3J and 3K are supplemental 2A, 2B, and 2C.

FIG. 4A shows SEM images displaying micropores (<10 μm and 100-200 μm range) of CO60HC10 scaffolds (1, 8, 15, 22, and 29 days) in different magnifications (100 μm, 500 μm, 1 mm). FIG. 4B shows a micro CT image of the CO60HC10 scaffold.

FIG. 5A shows XRD patterns of (a) PCL, (b) PCL/Collagen, and (c) PCL/Collagen/in situ HAPclay. FIG. 5B shows FTIR spectrum of PCL, PCL/Collagen, and PCL/Collagen/in situ HAPclay. FIG. 5C and FIG. 5D show DSC thermograms of CL60HC10 scaffolds before and after immersion in synovial fluids. Heating cycle (FIG. 5C). Cooling cycle (FIG. 5D). FIG. 5E illustrates a pin for creating a hole in the scaffold and schematic scaffold; FIG. 5F shows a 3D model of the scaffold with pin removed. FIG. 5G shows a schematic illustrating fiber orientation in a scaffold of the disclosure. FIG. 5G1 illustrates a parallel orientation, and FIG. G2 illustrates a Zig-Zag orientation. FIG. 5H shows SEM micrographs showing micropores (<10 μm and 100-200 μm range) of CO60HC10 scaffolds (with sutures) degradation in Day 0 and Day 8 in different magnifications (500 μm, 100 μm, 20 μm). FIG. 5I) shows tensile mechanical properties: representative stress-strain curves obtained for CO60HC10 with sutures with various patterns.

FIG. 6A shows live-dead imaging performed on hMSCs cultured on three different scaffolds (PCL, PCL/Collagen, and PCL/Collagen/in situ HAPclay). The viability of hMSCs was evaluated at four distinct time points (1, 3, 7, and 10 days). Green stain indicates live cells and red stain indicates dead cells-scale bar: 100 μm. FIG. 6B shows the number of live cell aggregates per 650×650 μm (with sutures), number of live cell aggregates per 650×650 μm (without sutures). FIG. 6C shows an illustration of the corresponding pathway for the collagen expression and YAZ and TAZ activation.

FIG. 7A and FIG. 7B show immunofluorescence staining of nestin, DAPI, collagen type I, and CD44 was observed in hMSCs grown on three different scaffolds (PCL, PCL/Collagen, and PCL/Collagen/in situ HAPclay) at day 10, where FIG. 7A shows scaffold without sutures and FIG. 7B shows scaffold with sutures. Scale bar: 100 μm.

FIG. 8A to FIG. 8C show immunofluorescence staining of CD31 (FIG. 8A), YAP (FIG. 8B), and TAZ (FIG. 8C) that was observed in the coculture of HUVECs and hMSCs grown on three different scaffolds (PCL, PCL/Collagen, and PCL/Collagen/HAPclay), at day 10 in scaffolds without sutures (w/o/s) and scaffolds with sutures (w/s). Scale bar: 100 μm.

FIG. 9 shows steps of a scaffold/IT Band technique-based surgery. Panel A1 shows 1 cm strip of IT band left attached distally. Panel A2 shows routing of IT band under LCL for modified Lemaire technique. Panel A3 shows planned drilling of 5 mm tunnel near anterior/distal footprint. Panel A4 shows the sliding of a scaffold of the disclosure over the IT band. Panel A5 shows a reamed tibial tunnel. Panel A6 shows the scaffold as placed in the knee.

FIG. 10A and FIG. 10B show a flowchart of a surgical method according to the disclosure.

Various embodiments of the present invention will be described in detail with reference to the figures, wherein like reference numerals represent like parts throughout the several views of various embodiments. Reference to various embodiments does not limit the scope of the invention. Figures represented herein are not limitations to the various embodiments according to the invention and are presented for exemplary illustration of the invention.

### DETAILED DESCRIPTION

A number of terms are introduced below, which are used to describe the invention of the present disclosure. In instances where a technical or scientific term is not specifically defined herein, they will have the same meaning as commonly understood by one of ordinary skill in the art. For example, any nomenclatures used in connection with and techniques of tissue engineering and surgical ligament and tendon repair described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein. It is to be understood that all terminology used herein is for the purpose of describing particular embodiments only and is not intended to be limiting in any manner or scope.

As used herein, the term “bioceramic” refers to a biocompatible ceramic composition that is resorbable by the body (i.e., eventually absorbed by and, in embodiments, replaced by the body after they have assisted repair; see also biodegradable) and can include one or more of mono-, di-, tri-, [alpha]-tri-, [beta]-tri-, and tetra-calcium phosphate, hydroxyapatite, calcium sulfates, calcium oxides, calcium carbonates, magnesium calcium phosphates.

As used herein, the term “biocompatible” refers to materials that interact with the body without an undesirable effect.

As used herein, the term “biodegradable” refers to materials which can be metabolized by the body, whether enzymatically, chemically, or by other in vivo degradation processes, and includes bioabsorbable reinforcement fibers with tailored rates.

As used herein, the term “clay” or “clay minerals” refer to fine-grained geologic material that develops plasticity when wet, but harden upon drying or firing. Clay or clay minerals may be, e.g., 2:1 clays, i.e., having two tetrahedral sheets of silica sandwiching a central octahedral sheet of alumina. Example clays include smectite, montmorillonite (e.g., Na-MMT, Ca-MMT), bentonite, beidellite, hectorite, those modified with amino acids (e.g., 5-aminovaleric acid), and mixtures or blends thereof, to enhance scaffold mechanical and biological properties.

As used herein, the term “composite” or “composite material” refers to a material that is produced from two or more constituent materials, e.g., a clay, a polymer, a polypeptide, and a calcium-based mineral in the present disclosure.

As used herein, the term “controlled release” and “sustained release” refer to control of the rate of release, quantity released, or combination thereof of an agent (e.g., drug, therapeutic agent, etc.). A controlled release can be continuous or discontinuous, linear or non-linear.

As used herein, the term “drug” refers to a substance intended for use in the cure, mitigation, prevention, and/or treatment of a disease, disorder, injury, or other condition in a human and/or non-human animal species.

As used herein, the term “effective amount” or “therapeutically effective amount” refers to the amount of an additional ingredient added that is sufficient to achieve its specific purpose(s). E.g., for a drug, to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). As used herein, the term “prophylaxis” refers to preventing or reducing the progression of a disorder, either to a statistically significant degree or to a degree detectable by a person of ordinary skill in the art. An “effective amount” of some additional ingredients, such as a growth factor, may enhance and ameliorate a positive characteristic, such as growth or healing of a repaired ligament (in contrast to reducing or ameliorating a disorder).

As used herein, the term “polymer” refers to a molecular complex comprised of more than ten monomeric units and generally includes, but is not limited to, homopolymers, copolymers, such as for example, block, graft, random and alternating copolymers, terpolymers, and higher “x”mers, wherein “x” is between 4 and 100, and further including their analogs, derivatives, combinations, and blends thereof. Furthermore, unless otherwise specifically limited, the term “polymer” shall include all possible isomeric configurations of the molecule, including, but are not limited to isotactic, syndiotactic and random symmetries, and combinations thereof. Furthermore, unless otherwise specifically limited, the term “polymer” shall include all possible geometrical configurations of the molecule.

As used herein, the term “polypeptide” refers to natural, modified, or recombinant collagen, collagen mimetic, or related peptides, or gelatin, which contribute to, e.g., scaffold structure, degradation profile, and/or biological integration.

As used herein, the term “peptide” refers to short chains of amino acids linked by peptide bonds that exhibit characteristics apart from that of collagen, a modified collagen, a collagen mimetic peptide, a collagen-related peptide, or otherwise polypeptide that displays collagen-like characteristics. An example is a therapeutic peptide.

As used herein, the term “amino acid” refers broadly to any molecule comprising at least one amino group (—NH₂), at least one carboxylic acid group (—COOH), and a side chain (R group) attached to a central carbon atom (the α-carbon), or any structurally analogous derivative thereof. The term “amino acid” encompasses the following:

Natural amino acid: One of the twenty genetically-encoded (“standard” or “canonical”) α-amino acids found in proteins produced by ribosomal translation in biological organisms, including glycine, alanine, valine, leucine, isoleucine, proline, phenylalanine, tryptophan, methionine, serine, threonine, cysteine, tyrosine, asparagine, glutamine, aspartic acid, glutamic acid, lysine, arginine, and histidine.

Unnatural amino acid: any amino acid analog or derivative not naturally encoded by the genetic code, whether found in nature (e.g., ornithine, norvaline) or generated synthetically in a laboratory.

Non-canonical amino acid: any amino acid other than the twenty standard amino acids as defined above, including amino acids that may be incorporated into proteins in organisms via expanded genetic codes, post-translational modifications, or translational misincorporation, for example, selenocysteine or pyrrolysine.

Non-proteinogenic amino acid: any amino acid that is not used directly in the genetic coding of proteins, but may occur naturally or be synthetically produced, including but not limited to β-alanine, γ-aminobutyric acid (GABA), 2-aminopimelic acid, 4-(4-aminophenyl) butyric acid, and others.

Synthetic amino acid: any amino acid or amino acid mimic, regardless of chirality, produced by artificial chemical synthesis, which may incorporate unique or altered functional groups, cyclic or aromatic side chains, or backbone modifications. This includes, e.g., D-amino acids, α,β-diamino acids, and amino acids bearing side-chain or backbone alterations not found in nature.

Modified amino acid: Any of the above amino acids which have been further altered chemically, such as by addition, deletion, or substitution of functional groups, post-translational modification (e.g., phosphorylation, methylation), or conjugation to a chemical moiety.

Combinations and derivatives thereof: Any mixture, blend, or derivative comprised of two or more of the above, as well as amino acids incorporated within oligomers, peptides, or conjugated to the clay or scaffold materials.

This definition of “amino acid” covers amino acids with α, β, γ or further-extended backbone lengths, any side chain or R group, any optical isomer, and any degree of chemical or physical modification, provided the amino acid moiety is capable of reacting with, binding to, or otherwise modifying the clay or scaffold as described in the invention.

As used herein, the term “scaffold” refers to three-dimensional matrix engineered to support desirable cellular interactions to contribute to the formation of new functional tissues for medical purposes.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the term “sustained release” refers to the continual release of an additional agent (e.g., drug, therapeutic agent, or combination thereof) over a period of time.

As used herein, “therapeutic agent” refers to any compound or composition of matter which, when administered to an organism (human or nonhuman animal) induces a desired pharmacologic, immunogenic, and/or physiologic effect by local and/or systemic action. The term therefore encompasses those compounds or chemicals traditionally regarded as drugs and biopharmaceuticals including molecules such as proteins, peptides, hormones, nucleic acids, and the like. The term “therapeutic agent” includes compounds or compositions for use in all of the major therapeutic areas including, but not limited to, anti-infectives such as antibiotics and antiviral agents; analgesics and analgesic combinations; local and general anesthetics; anti-inflammatory agents; hormones; plant extracts, bone growth stimulants and bone resorption inhibitors; proteins, peptides, and fragments thereof (whether naturally occurring, chemically synthesized or recombinantly produced).

As used herein, the term “therapeutic effect” means any improvement in the condition of a subject, human or animal, treated according to the subject method, including obtaining a preventative or prophylactic effect, or any alleviation of the severity of signs and symptoms of a disease, disorder, injury, or other condition which can be detected by means of physical examination, laboratory or instrumental methods.

As used herein, the terms “treat” and “treating” refer to: alleviating the severity of signs and symptoms of a disease, disorder, injury, or other condition; or inhibiting a disease, disorder, injury, or other condition in an animal or human that may be predisposed to the disease, disorder and/or other condition.

As used herein, the term “collagen” refers to a protein component of an extracellular matrix having a tertiary structure that includes polypeptide chains intertwining to form a collagen triple helix or having a characteristic amino acid composition comprising Gly-X-Y repeat units, or a fragment thereof. Although the present invention is described with reference to type I collagen, the term “collagen” as used herein refers to any one of the known collagen types, including collagen types I through XXIX, as well as to any other collagens.

As used herein, the term “modified collagen” refers to collagen that has been chemically modified by such means as phosphorylation, fluorination, halogenation (e.g., chlorination), sulfonation, hydroxylation, and/or cross-linking. Modified collagen also refers to collagen that has been partially (acid or base) hydrolyzed to form gelatin (Type A or Type B, respectively).

As used herein, the terms “collagen mimetic peptide” (CMP) and “collagen-related peptide” refer to a peptide that is able to form a collagen triple helical structure and physically interacts with a collagen polypeptide. In general a CMP binds collagen with high affinity. Example include (Gly-Pro-Pro), and (Gly-Pro-Hyp), trimer repeats. Collagen mimetic peptides and collagen-related peptides include homotrimeric forms, as well as heterotrimeric forms.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art of this disclosure.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

Recitation of numeric and compositional ranges (e.g., “between about 5% and about 20% by weight” and “pore size at least 200 nm”) are intended to include all intermediate values and subranges. These are only examples of what is specifically intended, and all possible combinations of numerical values between and including the lowest value and the highest value enumerated are to be considered to be expressly stated in this disclosure.

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used and may or may not reflect the final percentage of the active, wherein the final percentage of the active is dependent on the weight percent of active in the raw material.

Furthermore, when “about”, “approximately” and/or “substantially” is/are utilized to describe a value, this is meant to encompass minor variations (up to +/−10%) from the stated value. Where no stated value is provided, an element described as “substantially” means at least about 60%, 70%, 80%, 90%, 95%, 99%, or more of the element, as is logically coherent within in the context. Unless specifically stated to the contrary, for ranges specified using “about” language, the about applies to both ends of the recited range whether specified or not. For example, “between about 10 mM and 10 μM” is equivalent to “between about 10 mM and about 10 μM”.

As used herein, the terms “comprising”, “having”, “including”, and “containing” are to be construed as open-ended terms (i.e., meaning “including, but not limited to”) unless otherwise noted.

When introducing elements of the present disclosure or the aspects and embodiment thereof, the articles “a,” “an,” and “the” are intended to mean that there are one or more of the elements. Similarly, the adjective “another,” when used to introduce an element, is intended to mean one or more elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

As used herein, “optional” or “optionally” means that the subsequently described event or circumstance may or may not occur, and that the description includes instances where said event or circumstance occurs and instances where it does not.

Moving to the specifics of the subject matter, disclosed herein are scaffolds created by incorporating a polymer, polypeptide, clay, and calcium-based mineral into a composite, which composite is reinforced with a reinforcement structure (e.g., suture material). The scaffold also includes a through-hole to place repair ligament or tendon tissue (e.g., an iliotibial band autograft for ACL repair), an allograft, or other natural (non-ligament or tendon tissue) or non-natural (e.g., synthetic internal brace) analog, to enable intimate contact between replacement tissue and the bioactive scaffold. In embodiments, the clay is mineralized in situ with a calcium-based mineral (e.g., hydroxyapatite) within clay galleries, optionally after amino acid modification (e.g., 5-aminovaleric acid), enhancing mechanical integrity and cellular response, while a polypeptide (e.g., collagen or gelatin) modulates degradation and absorption characteristics to support ligament formation. The reinforcement structure can be arranged in parallel, spiral/zig-zag, or combined orientations to tailor tensile behavior and intraoperative handling. During an exemplary ACL procedure, femoral and tibial tunnels are drilled, and the scaffold and graft are drawn through; the bioabsorbable reinforcement maintains scaffold integrity under traction and assists passage through tunnels.

In addition to mechanical properties and degradation properties, scaffold absorption properties are important as they need to be able to quickly absorb patient blood that can be injected into it by a surgeon affecting the repair. The hole in the center of the scaffold allows for the placement of replacement tissue (e.g., IT band) such that the tissue is covered by the bioactive scaffold, creating an intimate contact between the two. This allows for better integration of the repair tissue and the cells and tissue growing in the scaffold. During an exemplary ACL repair, tunnels are drilled through the bone in the knee, and the scaffold and the IT band are pulled through the tunnel. The degradable suture reinforcement of the scaffolds maintains the integrity of the scaffold during surgery and is used to pull the scaffold through the tunnel.

The disclosed scaffolds address key hurdles in ligament and tendon repair by combining: (i) robustness in synovial fluid with tunable biodegradation, (ii) high blood and fluid absorption with retention under saline irrigation, and (iii) mechanical properties suitable for surgical manipulation and early function. Representative embodiments provide compressive moduli in the range of approximately 0.8-3.8 MPa pre-degradation and tensile moduli of approximately 24-27 MPa depending on reinforcement orientation, while achieving high absorption (up to about 85% by weight) and controlled degradation over clinically relevant timeframes in synovial fluid. These features provide procedural durability and a favorable biological milieu for repair and regeneration.

In one aspect, a composition is provided, the composition including a composite and a bioabsorbable reinforcement structure, the composite itself including a biocompatible clay (e.g., smectite or montmorillonite); a polymer (e.g., PCL or PLGA); a polypeptide (e.g., collagen or gelatin); and a calcium-based mineral (e.g., hydroxyapatite, in situ mineralized within the clay); wherein the composite forms a three-dimensional scaffold with a hole formed therethrough along an axis of the scaffold; and the bioabsorbable reinforcement structure is located on an outer surface of the scaffold, an inner surface of the scaffold, within the scaffold, or combinations thereof, and may be oriented, e.g., parallel, spiral/zig-zag, or in combination.

In another aspect, a method for preparing a composition (composite and one or more bioabsorbable reinforcement structures) of the disclosure is provided, including the steps of dissolving the clay in an aqueous solvent to form a dissolved clay; optionally modifying the clay with an amino acid; adding a calcium-based mineral (precursor) to the dissolved clay under controlled pH and temperature to form a mineralized clay precipitate; separating, drying and grinding the mineralized clay precipitate to form a mineralized clay powder; dissolving the polymer in a solvent to form a dissolved polymer; adding the polypeptide to the dissolved polymer to form a polymer and polypeptide mixture; adding the mineralized clay powder to the polymer and polypeptide mixture to form a clay, polymer, and polypeptide mixture; adding a bioabsorbable reinforcement structure within a mold of a desired three dimensional shape; adding the clay, polymer, and polypeptide mixture to the mold; placing an occluding device or stem through the clay, polymer, and polypeptide mixture to form a hole in the composite once the mixture is in solid form and the occluding device or stem is removed; freeze extracting or freeze gelating (i.e., using freeze extraction or freeze gelation protocols on) the clay, polymer, and polypeptide mixture; and forming a scaffold in the desired three-dimensional shape with a hole therethrough and a bioabsorbable reinforcement structure, once removed from the mold and removing the stem. In some embodiments the clay, polymer, and polypeptide mixture is 3-D printed to the desired shape. In some embodiments, the clay, polymer, and polypeptide mixture is 3-D printed in the absence of an occluding device.

Additional disclosure related to each aspect and its embodiments is provided below, including detailed compositions, reinforcement configurations, absorption, degradation, and mechanical data, cell and co-culture results, and surgical workflows with intraoperative customization, as shown in the figures and examples.

### Scaffolds

The present disclosure provides scaffolds comprising a biocompatible clay (including amino acid-modified forms such as 5-aminovaleric acid), a polymer, a polypeptide, a calcium-based mineral, and a bioabsorbable reinforcement structure, as well as methods for making them. The scaffold material may also include additional agents. In embodiments, the clay is in situ mineralized with hydroxyapatite (HAPclay) using controlled biomineralization in clay galleries to enhance mechanical strength and biological activity.

The scaffolds can be prepared in any suitable shape and size. In some embodiments, have porosities with at least one dimension that is at least 200 nanometers, and in some embodiments at least 400 nanometers, or at least 500 nanometers. In some embodiments, the scaffolds have porosities in at least two dimensions that are at least 200 nanometers, at least 400 nanometers, or at least 500 nanometers. In some embodiments the scaffolds have porosities in all three dimensions that are at least 200 nanometers, at least 400 nanometers, or at least 500 nanometers. These porosities are verified using scanning electron microscopy imaging or micro tomography x-ray scanning. The purpose of these porosities is to allow for fluid flow, cell growth and tissue regeneration and the pore to solid volume in the scaffold can exceed 80%.

In some embodiments, the scaffolds have at least two dimensions (e.g., diameter and length) that are, each separately, between about 1 millimeter and about 50 millimeters, or between 1 millimeter and about 45 millimeters, or between about 1 millimeter and 40 millimeters, or between about 1 millimeter and 35 millimeters, or between about 1 millimeter and 30 millimeters, or between about 1 millimeter and about 25 millimeters, or between about 1 millimeter and about 20 millimeters, or between about 3 millimeters and about 50 millimeters, or between about 3 millimeters and about 45 millimeters, or between about 3 millimeters and about 40 millimeters, or between about 3 millimeters and about 35 millimeters, or between about 3 millimeters and about 30 millimeters, or between about 3 millimeters and about 25 millimeters, or between about 3 millimeters and about 20 millimeters, or between about 5 millimeters and about 50 millimeters, or between about 5 millimeters and about 45 millimeters, or between about 5 millimeters and about 40 millimeters, or between about 5 millimeters and about 35 millimeters, or between about 5 millimeters and about 30 millimeters, or between about 5 millimeters and about 25 millimeters, or between about 5 millimeters and about 20 millimeters, or between about 10 millimeters and about 50 millimeters, or between about 10 millimeters and about 45 millimeters, or between about 10 millimeters and about 40 millimeters, or between about 10 millimeters and about 35 millimeters, or between about 10 millimeters and about 30 millimeters, or between about 10 millimeters and about 25 millimeters, or between about 10 millimeters and about 20 millimeters, or between about 15 millimeters and about 50 millimeters, or between about 15 millimeters and about 45 millimeters, or between about 15 millimeters and about 40 millimeters, or between about 15 millimeters and about 35 millimeters, or between about 15 millimeters and about 30 millimeters, or between about 15 millimeters and about 25 millimeters, and other sizes suitable for ligament or tendon repair.

The scaffolds can be prepared in any desired shape. For example, the scaffolds can be prepared with straight edges or rounded edges. The scaffolds can be in the shape of a sphere, a cube, a cylinder (e.g., circular cylinder, elliptical cylinder, etc), or any polygon. In the case of a polygon, the polygon can have any of its sides straight or rounded. To the extent that a scaffold has a flat base (e.g., not a sphere), an edge can be at a right angle from the base, or at an oblique angle from the base. Edges for a scaffold need not be at the same angle, e.g., a cylindrical scaffold can have one edge (in side view) at a right angle, and the other edge (in side view) at an oblique angle; or both edges can be at different oblique angles or both right angles, etc.

A distinguishing feature of the scaffolds is the axial through-hole, which is dimensioned to receive a ligament, tendon, autograft, allograft, or synthetic internal brace, enabling intimate contact between living tissue and bioactive matrix. This configuration ensures biological integration, rapid blood and fluid absorption, and functional anchoring during repair; clinical results support improved healing and post-operative support.

The scaffolds are reinforced by bioabsorbable structures which may include monofilament or polyfilament suture threads, braid, or mesh, arranged in parallel, spiral, zig-zag, or combined orientations. The reinforcement structure location (outer surface, inner surface, within scaffold, or combinations) and pattern directly influence tensile behavior, handling, and surgical deployment, as described herein. Tensile modulus can be tuned from about 24 MPa to 27 MPa or more, depending on the configuration.

Polymers can include, e.g., polycaprolactone (PCL), PLGA, polylactide, chitosan, natural or synthetic blends, and polypeptides (collagen, gelatin, collagen mimetic, etc.), with customized ratios for optimal degradation, handling, and cellular compatibility; exemplary compositions (e.g., CO40HC5, CO60HC10, CO70HC12) are detailed in Table 1 and corresponding data. Collagen content can range from, e.g, 40-70%, and in situ HAPclay from, e.g., 5-12%, modulating degradation and mechanical response.

Scaffold surfaces and matrix can additionally include, impregnate, or coat biological agents such as growth factors, cytokines, stem cells (including autologous MSCs, HUVECs), antibiotics, proteins, or gene therapy constructs, at concentrations and locations enabling sustained release and tailored biological effect. Integration of patient-derived cells, or custom agents, is supported by, e.g., soaking, injection, or direct scaffold loading.

The scaffolds can be sterilized and delivered as ready-to-use constructs or as kits with provision for further customization. The scaffolds can facilitate or assist the generation of tissue (e.g., ligament or tendon tissue), provide a vehicle for the delivery of various optional agents, and in embodiments, provide hierarchical structure for tissue to regenerate on or within. The scaffolds can allow for the use of the patient's own cells (autologous treatment) for tissue regeneration. The generation of tissue on, over, and/or around the scaffolds can be seen based on the formation of an extracellular matrix.

### Clay

The scaffolds described herein include a clay component. Suitable clays are those which are biocompatible and which, when incorporated into the composite, provide a beneficial impact on mechanical properties, cellular interactions, or degradation rates. Examples include nanoclays, which are (natural or synthetic) silicate clays. These silicates can be incorporated into a polymeric matrix to control the physicochemical properties of a nano-composite matrix. For example, for the polymer polycaprolactone (PCL), adding nanoclay increases PCL degradation rate, and montmorillonite (MMT) nanoclay-enriched PCL scaffolds support hMSCs adhesion and proliferation.

The scaffolds of the disclosure include a (nano) clay. Suitable clays for forming the scaffolds can include, but are not limited to, smectite clay minerals. In some embodiments of the compositions, the biocompatible clay includes bentonite, beidellite, hectorite, montmorillonite, nontronite, saponite, Kelocyte, magnesium aluminum borosilicate clay, or other silicates or combinations thereof. Reference to different species of clays includes the various types of that species, e.g., bentonite encompasses sodium bentonite, calcium bentonite, and potassium bentonite. Montmorillonite clay includes sodium montmorillonite and calcium montmorillonite, as well as montmorillonite clays with other cations. Some embodiments include more than one clay, which clays can exist as a mixture of clays, or, e.g., disposed in discrete regions or layers of a scaffold.

In certain embodiments, the clay is modified prior to mineralization, for example by treatment with amino acids such as 5-aminovaleric acid. Amino acid modification may be accomplished by contacting the clay with an aqueous amino acid solution at controlled temperature and pH, as illustrated in the “Modification of MMT clay” section, below. This modification is found to enhance dispersion within the composite, improve mechanical integrity, and facilitate cell attachment.

The clay is subsequently mineralized in situ by incorporation of a calcium-based mineral, such as hydroxyapatite. In situ mineralization may occur within the clay galleries by sequential mixing with calcium and phosphate precursors, resulting in hybrid HAPclay capable of mimicking native tissue biomineralization processes and contributing to the scaffold's mechanical and biological performance.

In some embodiments, the proportion of clay in the scaffold can range from about 1% to about 20% by weight, though the precise amount may be optimized for the intended use, polymer and polypeptide content, and targeted properties. The clay component can further influence pore structure, absorption rate, surface charge, and eventual resorption kinetics in vivo.

In some embodiments, the clay can comprise between about 1 wt. % to about 30 wt. % of the scaffold. In some embodiments, the clay can comprise between about 2 wt. % to about 25 wt. % of the scaffold. In some embodiments, the clay can comprise between about 5 wt. % to about 30 wt. % of the scaffold. In some embodiments, the clay can comprise between about 1 wt. % to about 25 wt. % of the scaffold. In some embodiments, the clay can comprise between about 1 wt. % to about 20 wt. % of the scaffold. In some embodiments, the clay can comprise between about 5 wt. % to about 25 wt. % of the scaffold. In some embodiments, the clay can comprise between about 5 wt. % to about 20 wt. % of the scaffold. In some embodiments, the clay can comprise between about 8 wt. % to about 20 wt. % of the scaffold. In some embodiments, the clay can comprise between about 8 wt. % to about 25 wt. % of the scaffold. In some embodiments, the clay can comprise between about 15 wt. % to about 22 wt. % of the scaffold, as well as other clay wt. % suitable for scaffolds of the disclosure.

In some embodiments, the proportion of clay in the scaffold can range from about 1% to about 20% by weight, though the precise amount may be optimized for the intended use, polymer and polypeptide content, and targeted properties. The clay component can further influence pore structure, absorption rate, surface charge, and eventual resorption kinetics in vivo.

Experimental data described herein indicate that amino acid-modified and in situ mineralized clays result in improved cell compatibility, rapid infiltration, and enhanced tenogenic differentiation, supporting use for ligament or tendon regeneration.

### Polymer

The compositions (and resulting scaffolds) of the disclosure include a polymer. The compositions can include more than one polymer. Preferably the polymer is biocompatible. In some embodiments, the polymer can be biodegradable.

Suitable polymers for use in the scaffolds include any polymeric material without limitation so long as it possesses the necessary biocompatible and/or biodegradable properties. Preferred polymers include those of natural and synthetic origins, and blends, combinations, or mixtures of the same, which can be formed into copolymers, terpolymers, or “x” mers.

Examples of natural polymers include, but are not limited to, proteins and polysaccharides, which can be used individually, in blends, combinations and/or mixtures. In some embodiments, the scaffolds include natural polymers including one or more of albumin, alginate, cellulose (which is inclusive of regenerated cellulose), hyaluronic acid, fibrin, fibronectin, keratin, and laminin, chitin, chitosan, collagen, gelatin, heparin, and other naturally occurring polymers such as regenerated silk or polysaccharide, and/or blends, combinations, or mixtures of the same.

Examples of synthetic polymers include, but are not limited to, poly(amino acids), polyanhydrides, polyesters, poly(alpha-hydroxy acids), poly(lactones), poly(orthocarbonates), poly(orthoesters), poly(phosphoesters), or polyphosphazenes, which can be used individually, in blends, combinations and/or mixtures. In some embodiments, the synthetic polymers include polycaprolactone (PCL), poly(delta-valerolactone), poly(1,5-dioxepan-2-one), poly(epsilon-aprolactone), poly(ester urethane) (PEU), polygalactouronic acid, poly(gamma-butyrolactone), polyglycolic acid (PGA), poly(alpha-hydroxy acids), polyhydroxyalkanoate (PHA), polyhydroxybutyric acid, poly(3-hydroxybutyrate-co-3-hydroxyvalerate (PHBV), polylactic acid (PLA) (e.g., poly(DL-lactic acid) and poly(L-lactic acid)), copolymers of lactic acid-glycolic acid such as poly(lactic-co-glycolic acid) or poly(lactide-co-glycolide) (PLGA), poly(lactic acid-co-caprolactone) (PLCL), poly(para-dioxanone) (PPDO), poly(trimethylene carbonate), poly-8-valerolactone, or blends, combinations, and mixtures of the same. In some embodiments, the polymer is polycaprolactone (PCL). PCL is known for its favorable degradation profile and mechanical strength. In some embodiments, the polymer is polylactic acid (PLA), polyglycolic acid (PGA), poly(lactic-co-glycolic acid) (PLGA), chitosan, and blends or copolymers thereof.

In some embodiments, the polymer includes albumin, alginate, cellulose, chitin, chitosan, collagen, gelatin, heparin, regenerated silk polymer, polysaccharide, poly(amino acid), polyanhydride, polyester, poly(alpha-hydroxy acid), poly(lactone), poly(orthocarbonate), poly(orthoester), poly(phosphoester), polyphosphazenes, blend, mixture, combination thereof.

In some embodiments, the polymer includes polyacrylonitrile, polycaprolactone, poly(delta-valerolactone), poly(1,5-dioxepan-2-one), poly(epsilon-aprolactone), poly(ester urethane), polygalactouronic acid, poly(gamma-butyrolactone), polygly colic acid, poly(alpha-hydroxy acids), polyhydroxyalkanoate, polyhydroxybutyric acid, poly(3-hydroxybutyrate-co-3-hydroxyvalerate, polyimide, polylactic acid, poly(lactic-co-gly colic acid), poly(lactic acid-co-caprolactone), poly(trimethylene carbonate), poly-8-valerolactone, or blends, combinations, and mixtures of the same. In some embodiments, the polymer includes chitosan-polygalactouronic acid, polycaprolactone, or a blend, combination, or mixture thereof, and in some embodiments, the polymer is polycaprolactone (PCL).

As described elsewhere herein, blending PCL with polylactide, PLGA, or chitosan can further optimize degradation rates and scaffold flexibility. The polymer component is found to regulate the overall scaffold integrity during implantation and subsequent tissue regeneration, and may be tailored to match the required resorption period for ligament or tendon healing.

Polymer content in exemplary compositions ranges from about 15% to about 60%, or about 20% to about 55%, by weight, with certain specific ratios detailed in Table 1. Mixtures of PCL/polymer blends display enhanced integration with clay and polypeptide phases, as well as improved cell attachment over PCL alone.

Polymers may be dissolved in an appropriate solvent (e.g., dichloromethane, acetone) to form homogeneous solutions prior to mixing with clay and polypeptide, as described in the elsewhere herein. The molecular weight and blend composition of the polymer can be selected according to the intended stiffness, degradation profile, and process method (e.g., freeze extraction, 3D printing).

In some embodiments, the polymer matrix is designed to facilitate reinforcement fiber placement (parallel, spiral, zig-zag, etc.) as described below. The polymer phase can also act as a carrier or matrix for bioactive agents when loaded into the scaffold, including growth factors, stem cells, or other therapeutics.

The scaffold polymer can be sterilized using standard pharmaceutical or surgical practices without loss of key mechanical or biological properties, ensuring compatibility with clinical workflows.

### Polypeptide

The compositions (and resulting scaffolds) of the disclosure also include a polypeptide. The compositions can include more than one polypeptide. Suitable polypeptides for use in the scaffolds of the disclosure are those which contribute to, e.g., biocompatibility, scaffold structure, controlled degradation, and biological activity.

Polypeptides of the disclosure include, e.g., natural collagen, recombinant or synthetic collagen, collagen mimetic peptides, collagen-related peptide, and collagen-derivatives. Herein, the terms “collagen” or “collagen-like” refer to proteins or polypeptide chains which comprise Gly-X-Y triplet sequences with a minimum of three triplets in any of its three registers (that is . . . Gly-X-Y-Gly-X-Y-Gly-X-Y . . . , . . . Y-Gly-X-Y-Gly-X-Y-Gly-X . . . , or . . . X-Y-Gly-X-Y-Gly-X-Y-Gly . . . ), independently of the polypeptides forming trimers or proteins forming a triple helical structure or not. Thus, the definition of collagen or collagen-like domains refers to the occurrence of the repetitive sequence at the primary structure level, and bears no implications for the actual secondary, tertiary or quaternary structures of the polypeptide or protein containing it. This particular sequence enables collagen to form its characteristic triple-helical structure. The term “triplet” refers to a set of three amino acids as defined by the set Gly-X-Y, wherein X and Y can be any amino acid, or alternative triplets for collagen mimetics, collagen-related peptides and the like. In the present disclosure, the term “collagen” includes naturally occurring collagen, and fragments, domains, derivatives (i.e., modified), mimetics, variants, related and chemically modified compounds of said naturally occurring collagen. Preferably, the eukaryotic collagen or collagen-like domains of the invention will be capable of mediating one or more collagen activities, such as being able to bind to cell surface molecules such as integrin or fibronectin, or glycoproteins or proteoglycans, or will be derived from a eukaryotic collagen protein which is capable of mediating one or more such activities.

In some embodiments, the polypeptide includes collagen, a modified collagen (including gelatin), a collagen-related peptide, a collagen-mimetic peptide, or combinations thereof. In some embodiments, the polypeptide includes a modified collagen. In some embodiments, the polypeptide includes a collagen-related peptide or collagen-mimetic peptide, and in some embodiments, the polypeptide is collagen.

Generally, the polypeptide component may be derived from natural sources, produced recombinantly, or chemically modified to adjust degradation rate, mechanical properties, or cell-binding activity.

In some composition embodiments, the polypeptide content can range from about 40% to about 70% by weight, tuned to both mechanical requirements and cellular integration. Increased collagen ratios are shown to enhance scaffold degradation in synovial fluid, and also promote higher fluid and blood absorption rates (up to about 85% by weight).

In other embodiments, the polymer and polypeptide together comprise between about 10 wt. % to about 90 wt. % of the composite; or between about 10 wt. % to about 85 wt. % of the composite; or between about 10 wt. % to about 80 wt. % of the composite; or between about 10 wt. % to about 75 wt. % of the composite; or between about 10 wt. % to about 70 wt. % of the composite; or between about 10 wt. % to about 65 wt. % of the composite; or between about 10 wt. % to about 60 wt. % of the composite; or between about 10 wt. % to about 55 wt. % of the composite; or between about 10 wt. % to about 50 wt. % of the composite; or between about 10 wt. % to about 45 wt. % of the composite; or between about 10 wt. % to about 40 wt. % of the composite; or between about 10 wt. % to about 35 wt. % of the composite; or between about 15 wt. % to about 90 wt. % of the composite; or between about 15 wt. % to about 85 wt. % of the composite; or between about 15 wt. % to about 80 wt. % of the composite; or between about 15 wt. % to about 75 wt. % of the composite; or between about 15 wt. % to about 70 wt. % of the composite; or between about 15 wt. % to about 65 wt. % of the composite; or between about 15 wt. % to about 60 wt. % of the composite; or between about 15 wt. % to about 55 wt. % of the composite; or between about 15 wt. % to about 50 wt. % of the composite; or between about 15 wt. % to about 45 wt. % of the composite; or between about 15 wt. % to about 40 wt. % of the composite; or between about 10 wt. % to about 35 wt. % of the composite; or between about 20 wt. % to about 90 wt. % of the composite; or between about 20 wt. % to about 85 wt. % of the composite; or between about 20 wt. % to about 80 wt. % of the composite; or between about 20 wt. % to about 75 wt. % of the composite; or between about 20 wt. % to about 70 wt. % of the composite; or between about 20 wt. % to about 65 wt. % of the composite; or between about 20 wt. % to about 60 wt. % of the composite; or between about 20 wt. % to about 55 wt. % of the composite; or between about 20 wt. % to about 50 wt. % of the composite; or between about 20 wt. % to about 45 wt. % of the composite; or between about 20 wt. % to about 40 wt. % of the composite; or between about 10 wt. % to about 35 wt. % of the composite; or between about 25 wt. % to about 90 wt. % of the composite; or between about 25 wt. % to about 85 wt. % of the composite; or between about 25 wt. % to about 80 wt. % of the composite; or between about 25 wt. % to about 75 wt. % of the composite; or between about 25 wt. % to about 70 wt. % of the composite; or between about 25 wt. % to about 65 wt. % of the composite; or between about 25 wt. % to about 60 wt. % of the composite; or between about 25 wt. % to about 55 wt. % of the composite; or between about 25 wt. % to about 50 wt. % of the composite; or between about 25 wt. % to about 45 wt. % of the composite; or between about 25 wt. % to about 40 wt. % of the composite; or between about 25 wt. % to about 35 wt. % of the composite.

In some embodiments, the polymer and polypeptide are present in about a 100:1 ratio (where the ratio is written as polymer:polypeptide), or about a 90:1 ratio, or about a 80:1 ratio, or about a 70:1 ratio, or about a 65:1 ratio, or about a 60:1 ratio, or about a 55:1 ratio, or about a 50:1 ratio, or about a 45:1 ratio, or about a 40:1 ratio, or about a 35:1 ratio, or about a 30:1 ratio, or about a 25:1 ratio, or about a 20:1 ratio, or about a 15:1 ratio, or about a 10:1 ratio, or about a 9:1 ratio, or about a 8:1 ratio, or about a 7:1 ratio, or about a 6:1 ratio, or about a 5:1 ratio, or about a 4:1 ratio, or about a 3:1 ratio, or about a 2:1 ratio, or about a 1:1 ratio, or where the ratio is written as polypeptide:polymer), or about a 100:1 ratio, or about a 100:1 ratio, or about a 90:1 ratio, or about a 80:1 ratio, or about a 70:1 ratio, or about a 65:1 ratio, or about a 60:1 ratio, or about a 55:1 ratio, or about a 50:1 ratio, or about a 45:1 ratio, or about a 40:1 ratio, or about a 35:1 ratio, or about a 30:1 ratio, or about a 25:1 ratio, or about a 20:1 ratio, or about a 15:1 ratio, or about a 10:1 ratio, or about a 9:1 ratio, or about a 8:1 ratio, or about a 7:1 ratio, or about a 6:1 ratio, or about a 5:1 ratio, or about a 4:1 ratio, or about a 3:1 ratio, or about a 2:1 ratio.

The collagen (or polypeptide) may be dissolved in a suitable solvent (e.g., acetic acid, dilute HCl) before combining with polymer and mineralized clay, as described elsewhere herein. Lower molecular weight or partially hydrolyzed gelatin variants can be used to further modulate mechanical and resorption profiles.

As disclosed herein, collagen-rich scaffold variants provided improved cell compatibility and support robust human mesenchymal stem cell (hMSC) attachment, proliferation, and tenogenic differentiation.

The polypeptide phase can also function as a carrier for biologically active agents, including growth factors or cell adhesion peptides, which can be incorporated during scaffold preparation or loaded post-fabrication.

### Calcium-Based Minerals

The scaffolds described herein include a calcium-based mineral component incorporated into the composite. One suitable mineral is hydroxyapatite, which is structurally and chemically analogous to native bone mineral and supports osteoconduction as well as ligament/tendon repair.

In some embodiments, the calcium-based mineral comprises a bioceramic, which in some embodiments includes one or more of a mono-, di-, tri-, [alpha]-tri-, [beta]-tri-, tetra-calcium phosphate, hydroxyapatite, a calcium sulfate, a calcium oxide, a calcium carbonate, a magnesium calcium phosphate, or combinations thereof. In some embodiments, the calcium-based mineral is hydroxyapatite.

In some embodiments, the calcium-based mineral is incorporated by in situ mineralization within clay galleries. The in situ HAPclay process involves sequential treatment of amino acid-modified nanoclay with phosphate and calcium precursors under controlled pH and temperature to produce hybrid mineralized clay. This approach yields a composite with enhanced stiffness, cell compatibility, and scaffold integration, as described elsewhere herein.

The mineral content in scaffolds of some embodiments ranges from about 5% to about 12% by weight, with precise values optimized for specific combinations of polymer and polypeptide, desired degradation rate, and application (ACL, tendon, or bone repair).

In some embodiments, the calcium-based mineral comprises between about 0.1 wt. % to about 25 wt. % of the composite, or between about 0.1 wt. % to about 20 wt. % of the composite, or between about 0.1 wt. % to about 15 wt. % of the composite, or between about 0.5 wt. % to about 25 wt. % of the composite, or between about 0.5 wt. % to about 20 wt. % of the composite, or between about 0.5 wt. % to about 15 wt. % of the composite, or between about 1 wt. % to about 25 wt. % of the composite, or between about 1 wt. % to about 20 wt. % of the composite, or between about 1 wt. % to about 15 wt. % of the composite, or between about 2 wt. % to about 25 wt. % of the composite, or between about 2 wt. % to about 20 wt. % of the composite, or between about 2 wt. % to about 15 wt. % of the composite, or between about 3 wt. % to about 25 wt. % of the composite, or between about 3 wt. % to about 20 wt. % of the composite, or between about 3 wt. % to about 15 wt. % of the composite, or between about 5 wt. % to about 25 wt. % of the composite, or between about 5 wt. % to about 20 wt. % of the composite, or between about 5 wt. % to about 15 wt. % of the composite.

As described elsewhere herein, in situ mineralization leads to improved dispersion of hydroxyapatite within the composite, efficient pore formation, and better maintenance of mechanical properties over time, including in synovial fluid environments.

The calcium-based mineral also provides nucleation sites for cell attachment, enhances osteogenic potential, and may support rapid tissue integration in cases where tendon or ligament is grafted near bone. It can also act as a carrier or stabilizer for bioactive molecules (e.g., growth factors) when included within the composite matrix.

Other mineralization protocols, e.g., ex situ blending, surface coating, or post-fabrication impregnation, can be employed as alternative embodiments, though in situ methods are generally better suited for homogeneous distribution and reproducible performance.

### Bioabsorbable Reinforcement Structure

The scaffolds disclosed herein include at least one bioabsorbable reinforcement structure, which serves to enhance mechanical strength, facilitate surgical handling, and support tissue integration during and after ligament or tendon repair.

Such reinforcement structures can include, e.g., one or more monomer threads, polymer threads, bundled fibers, woven or non-woven meshes, braided constructs, or combinations thereof, according to the demands of intended surgical use and desired mechanical profile. An example of a “thread” includes any string-like material that is biocompatible and possesses the physical characteristics necessary for reinforcing the structure of the compositions (scaffolds) of the disclosure. While a suture is typically a thread that approximates and maintains tissues until the natural healing process has provided a sufficient level of wound strength or compresses blood vessels in order to stop bleeding, herein the suture thread helps maintain the structural integrity of a scaffold during its insertion procedure and for an amount of time suitable for the procedure being performed.

Sutures can be classified into one of two groups, absorbable and nonabsorbable. Absorbable sutures are temporary due to their ability to be “absorbed” or decomposed by the natural reaction of the body to foreign substances. An absorbable suture is one that loses its tensile strength a number of days, e.g., within 60 days, 45 days, 30 days, and the like. That is, different absorbable sutures have different resistance levels to absorption, and each can be formulated or treated in order to obtain a desired decomposition rate. Nonabsorbable sutures are, in like manner, sutures that are not dissolved or decomposed by the body's natural action. Such sutures are generally not naturally occurring materials, with the exception of silk; silk and nylon, while being classified as nonabsorbable, actually dissolve after a long period of time compared to that of other absorbable materials.

Sutures are manufactured with a wide variety of parameters. They can be monofilament or many filaments twisted together, spun together, or braided (polyfilament). They can also be dyed, undyed, coated, not coated.

Currently, sutures are designed to result in the most desirable effect for any given situation as determined by those administering the sutures. Taken into consideration in the manufacture and use of sutures are properties such as stress-strain relationship, tensile strength and rate of retention, flexibility, intrinsic viscosity, wettability, surface morphology, degradation, thermal properties, contact angle of knots, and elasticity.

Properties such as stress-strain relationship and tensile strength have a direct effect on how much force at a given rate the scaffold reinforced with suture thread or mesh made therefrom will be able to withstand before failure or breakage. For example, a sharp blow or forcing a scaffold through an undersized hole in bone or skin in an arthroscopic procedure would impose a fast rate of elongation; whereas, edema or hemorrhage in the surgical area could impose a slow rate of elongation. Surface morphology, the description and condition of the outer surface of the suture, can have an effect on how the scaffold in which the suture is located is affected when force is placed upon the suture/scaffold. For example, a braided suture can cause a type of sawing effect at the point of contact with the scaffold when moved.

Flexibility in relation to tensile strength is also of high priority in the choice of a suture or mesh made of suture-like materials. Some materials are braided polyfilaments as they can be excessively stiff for handling if formed into monofilaments of sufficient diameter and strength to hold the scaffold together while under mechanical stress. The rate of degradation is important due to the difference of required periods of time that a scaffold is needed to maintain its strength and placement within the surgical field (e.g., knee joint or shoulder joint).

Selection of the best reinforcement structure material for a given scaffold involves consideration of all of these factors to produce the most well suited scaffold.

Some important characteristics of suture material, mesh made therefrom, or other materials utilized as a reinforcement structure include tensile strength, as related to thread/suture size and as related to weight required to break a thread/suture or mesh; elasticity, or degree suture stretches and return to original length; memory or thread/suture/mesh stiffness, wherein high memory correlates to stiffness and difficult of handling; and tissue reactivity, such as the inflammatory response to the thread/suture/mesh. In general, favorable thread/suture/mesh characteristics include inertness, adequate tensile strength, flexibility, ease in handling, nonallergenic nature, resistance to infection, and absorbability.

Thread/suture/mesh material characteristics. Exemplary thread/suture/mesh materials for use as reinforcement structures include natural, absorbable and synthetic absorbable varieties. Examples of natural absorbable embodiments can include plain catgut, which is prepared from the submucous coat of sheep or cow intestine. Plain catgut is fairly rapidly hydrolyzed in tissues and loses its integrity within several days. Generally, it should only be used in tissues where fairly rapid hydrolysis is not a disadvantage. Catgut can have a variable resorption rate, and thus unpredictable tensile strength, and is associated with greater tissue reaction than synthetic materials. Chromic catgut has been treated with chromic acid salts to affect cross-binding, which delays hydrolysis. Generally, it should not be used where absorption progresses before healing is complete enough to restore adequate tensile strength. Absorption can be unpredictable, particularly in contaminated or infected wounds. Examples of synthetic, absorbable embodiments include Polyglecaprone 25 (Monocryl), polydioxanone (PDS), polyglactin-910 (Vicryl), polyglycolic acid (Dexon), and polycarbonate, which are braided or monofilament synthetic polymeric sutures that retains their integrity for a significant time in the body. They are degraded by hydrolysis in a slower and more predictable fashion than catgut.

In some embodiments, the bioabsorbable reinforcement structure includes a monofilament thread, a polyfilament thread, a mesh, or combinations thereof. In some embodiments the bioabsorbable reinforcement structure is a polyfilament thread. In some embodiments, the bioabsorbable reinforcement structure is a monofilament thread, while in some embodiments, the the bioabsorbable reinforcement structure is a mesh.

Orientation and placement of the one or more threads/sutures/woven or non-woven mesh/filaments/braided constructs within the scaffold is also an important consideration. The reinforcement structure(s) can be placed within the scaffold, on one or more surfaces of the scaffold, as well as being distributed within multiple scaffold zones, or combinations thereof. Reinforcement structures can be placed in an organized arrangement, randomly placed or combinations thereof, as well as oriented along one or more axes of a scaffold, oriented off axis (e.g., not along the X, Y or the Z axis of the scaffold, an example being placed diagonally through the scaffold material, or in a circular or spiral arrangement through the scaffold material).

In embodiments, the orientation of reinforcement is selected to optimize tensile performance, resilience to surgical loading, and positional stability during graft placement. Arrangements may include parallel orientation (fibers aligned along the axis of the through-hole), spiral orientation (fibers wrapped helically around the axis), zig-zag patterns traversing the scaffold in a serpentine fashion, or a combination of these. Experimental data as described herein demonstrate that zig-zag and combined patterns yield substantial tensile modulus and handling, retaining mechanical strength (up to about 27 MPa or more) even after soaking in synovial fluid.

In some embodiments, the bioabsorbable reinforcement structure is oriented parallel to the scaffold axis along which the through-hole is formed. In some embodiments, the parallel bioabsorbable reinforcement structure is located on an outer surface of the scaffold, an inner surface of the scaffold, within the scaffold, or combinations thereof. In some embodiments, the bioabsorbable reinforcement structure is oriented in a spiral pattern around the scaffold axis along which the hole is formed. In some embodiments with a spiral reinforcement structure, the spiral reinforcement structure is located on an outer surface of the scaffold, an inner surface of the scaffold, within the scaffold, or combinations thereof. In some embodiments, the reinforcement structure is oriented parallel to the scaffold axis along which the hole is formed and a second bioabsorbable reinforcement structure is oriented in a spiral pattern around the scaffold axis along which the hole is formed, each reinforcement structure located on an outer surface of the scaffold, an inner surface of the scaffold, within the scaffold, or combinations thereof.

In some embodiments, the bioabsorbable reinforcement structure includes a monofilament thread, polyfilament thread, or mesh, in which the monofilament thread, polyfilament thread, or mesh include a natural absorbable material, a synthetic absorbable material, or combinations thereof. In some embodiments that include a natural absorbable material, the natural absorbable material includes chromic gut, non-chromic gut, or combinations thereof. In some embodiments that include a synthetic absorbable material, the synthetic absorbable material includes Polyglycolic acid (Dexon), Polyglactin 910 (Vicryl), Poliglecaprone 25 (Monocryl), Polydioxanone (PDS II), Polyglyconate (Maxon), polydioxanone (PDS), and the like, or combinations thereof.

A bioabsorbable reinforcement can also acts as an anchor for autograft/allograft tissue or internal brace. Reinforcement material can be selected to degrade over a time period coordinated with scaffold resorption and tissue integration, ranging from weeks to several months depending on the polymer chemistry.

Reinforcement structures may further serve as carriers for bioactive agents-such as growth factors, antibiotics, or patient-derived cells-either by impregnation, surface coating, or integration during scaffold preparation.

### Additional Agents

The scaffolds can optionally contain any number of additional agents added to enhance biological, therapeutic, or mechanical properties. The additional agents can be naturally occurring or synthetic, organic or inorganic. Suitable additional agents, include, but are not limited to amino acids, anesthetics, antibiotics, anti-inflammatory agents (which antibiotic and anti-inflammatory agents may be incorporated, e.g., for infection prevention and management of tissue response; controlled and sustained-release formulations may also be achieved through polymer design or micro/nanoscale encapsulation), antiangiogenic agents, antibodies, anticoagulants, antineoplastic agents, antiviral agents, biomaterials, bone morphogenetic proteins, carbohydrates, cells, cytotoxic agents, cytokines, drugs, electrolytes, gene therapy constructs, growth factors (e.g., bone morphogenetic proteins, TGF-β, vascular endothelial growth factor), plant extracts, immunomodulators, inorganic materials, lipids, minerals, oligonucleotides, osteoblasts, osteoclasts, osteo stem cells, peptides, polypeptides, progenitors, proteins, stem cells (adult and/or embryonic), therapeutic agents, tissues, tissue or cell aggregates, vasoactive agents, and combinations thereof. Preferred proteins include bone morphogenetic protein (BMP), particularly BMP-2, BMP-7, and BMP-12. In some embodiments, the additional agent is an amino acid, anesthetic, antibiotic, antiangiogenic agent, antibody, anticoagulant, antineoplastic agent, antiviral agent, biomaterial, bone morphogenetic proteins, carbohydrate, cell, cytotoxic agent, drug, electrolyte, growth factor, immunomodulator, inorganic material, lipid, mineral, oligonucleotide, osteoblast, osteoclast, osteo stem cell, polypeptide, progenitor, protein, therapeutic agent, tissue, tissue or cell aggregate, vasoactive agent, and combinations thereof. IN some embodiments, the additional agent is one or more of a human osteoblast, non-human animal species osteoblast, amino acid, growth factor, bone morphogenic protein, and/or an adult stem cell.

As described elsewhere herein, inclusion of agents such as hMSCs and HUVECs greatly enhance cell compatibility, vascularization, proliferation, and expression of tenogenic (SCX, TNMD) and angiogenic (CD31, YAP/TAZ) markers.

Additional agents can be added in an effective amount, that is in an amount effective to achieve a specific purpose. For example, if an antibiotic additional agent is included in a composition of the disclosure, the antibiotic is preferably added at a sufficient concentration to prevent or eliminate infection of the repaired ligament or tendon.

It has been found that increased backbone length of amino acids can increase molecular interaction between polymer, amino acid and clay allowing for significant improvement in mechanical properties. Unnatural amino acids provide longer backbone chains and are thus candidates as modifiers. Thus, some embodiments of the invention include an amino acid with a carbon backbone chain length of at least five carbon atoms. Suitable amino acids have a carbon chain length of between one and about ten. In some embodiments, amino acids include but are not limited to, aminovaleric, amino caprylic, amino pimelic, 4-(4-Aminophenyl) butyric acids and combinations thereof.

The additional agents can be selected to impart particular functionalities or properties. For example, additional agents can be selected to affect and/or control the mechanical, biological and degradation properties of the scaffold. In another aspect of the invention, specific additional agents can be selected for the desired properties or effects and based on the patient. For example, in the case of a human patient, a human osteoblast can be used, whereas if the patient is a cow a bovine osteoblast can be used. Similarly, certain additional agents can be specifically tailored to the patient based on use of their own genetic and/or cellular materials, e.g., cell lines developed based on compatibility or directly from the patient's own genetic and/or cellular materials. In a preferred embodiment of the invention, the scaffolds and/or scaffold blocks can incorporate autologous treatments.

Any suitable amount of additional agents can be used in the scaffolds. The appropriate amount of an additional agent can be dictated by the patient's condition, age, size, general health, medical conditions, allergies, etc., and, in embodiments, is present in an effective amount. Generally, the additional agents will be included in an amount of between 0.01 wt. % and 50 wt. % of the composition. The additional agents can be part of the scaffolds (e.g., entrapment within polymer or polypeptide matrix), impregnated within the scaffolds, coat the scaffolds (e.g., surface adsorption or coating), or any combination thereof. The additional agents can be attached to, coat, and/or modify the clay, polymer, bioabsorbable reinforcement structure, polypeptide, or calcium-based mineral. In an aspect of the invention, when used to coat the scaffolds, the additional agents can be prepared in a solution and the scaffolds can be soaked in the solution. In some embodiments, the one or more additional agent(s) is released by a controlled release and/or sustained release.

Additional agents may be selected according to the intended repair tissue (ligament, tendon, bone) and desired clinical outcome, and may be tailored to the patient or application.

### Methods of Preparing the Scaffolds

The scaffolds of this disclosure are generally prepared through a multi-stage process designed to ensure precise control over composition, structure, and the integration of bioabsorbable reinforcement. Initially, the clay component, such as montmorillonite or bentonite, is dispersed in aqueous solution to achieve uniform mixing. In some embodiments, this clay undergoes amino acid modification, e.g., with 5-aminovaleric acid, by incubation at controlled pH and temperature; this treatment enhances both dispersion and future mineralization, providing beneficial properties to the final scaffold.

Following clay modification, the next step is in situ mineralization, where a calcium-based mineral, in embodiments hydroxyapatite, is formed within the clay galleries. This is accomplished by sequentially adding a phosphate source (such as NaHPO₄) and a calcium source (such as CaCl₂)) to the prepared clay under optimized conditions (approximately pH 7.5), producing a hybrid HAPclay. The resulting mineralized clay is then separated, dried, and ground to nanopowder.

Concurrently, the selected polymer, e.g., polycaprolactone (PCL), PLA, PLGA, or chitosan, is dissolved in a suitable solvent, while the polypeptide (such as type I collagen, gelatin, or a synthetic peptide) is dissolved separately in a suitable solvent, e.g., a dilute acid. The mineralized clay nanopowder is added to the polymer and polypeptide solutions, forming a homogeneous composite through mixing, sonication, or mechanical agitation. Ratios are carefully adjusted to match the targeted scaffold properties.

Integration of the bioabsorbable reinforcement, which may take the form of, e.g., suture(s) filaments, mesh(es), or braid(s), generally occurs during molding. The reinforcement can be introduced into the mold along specific orientations-parallel, spiral, zig-zag, or combinations thereof. Molds or 3D-printed fixtures are often used to set the geometry and position of the fibers, as well as to form the axial through-hole integral to the finished scaffold.

The composite mixture is cast into the mold and then solidified through freeze extraction, freeze drying, or gelation at controlled temperatures. In some embodiments, scaffolds are created using alternative techniques such as lyophilization or 3D printing, with or without occluding devices to create the passage for graft tissue. Biological agents, including cells, growth factors, or drugs, can be incorporated during mixing, after solidification via soaking or injection, or by surface functionalization as required for the intended clinical application.

Once solidified, the scaffold is carefully removed from the mold, and any occluding device is withdrawn to leave the central through-hole. Final sterilization is performed using accepted medical protocols, such as ethylene oxide or gamma irradiation. Throughout the process, the dimensions, porosity, reinforcement layout, and included agents are validated against design and experimental requirements. The described preparation may be adapted for laboratory-scale or continuous production, ensuring reproducibility and scalability for clinical use.

All steps are further detailed and validated within the Examples and supporting figures, which provide additional information on specific compositions, manufacturing parameters, and resulting scaffold performance.

In some embodiments, the scaffold has at least one dimension that is at least 5 millimeters. In some embodiments, the scaffolds have at least two dimensions (e.g., diameter and length) that are, each separately, between about 1 millimeter and about 50 millimeters, or between 1 millimeter and about 45 millimeters, or between about 1 millimeter and 40 millimeters, or between about 1 millimeter and 35 millimeters, or between about 1 millimeter and 30 millimeters, or between about 1 millimeter and about 25 millimeters, or between about 1 millimeter and about 20 millimeters, or between about 3 millimeters and about 50 millimeters, or between about 3 millimeters and about 45 millimeters, or between about 3 millimeters and about 40 millimeters, or between about 3 millimeters and about 35 millimeters, or between about 3 millimeters and about 30 millimeters, or between about 3 millimeters and about 25 millimeters, or between about 3 millimeters and about 20 millimeters, or between about 5 millimeters and about 50 millimeters, or between about 5 millimeters and about 45 millimeters, or between about 5 millimeters and about 40 millimeters, or between about 5 millimeters and about 35 millimeters, or between about 5 millimeters and about 30 millimeters, or between about 5 millimeters and about 25 millimeters, or between about 5 millimeters and about 20 millimeters, or between about 10 millimeters and about 50 millimeters, or between about 10 millimeters and about 45 millimeters, or between about 10 millimeters and about 40 millimeters, or between about 10 millimeters and about 35 millimeters, or between about 10 millimeters and about 30 millimeters, or between about 10 millimeters and about 25 millimeters, or between about 10 millimeters and about 20 millimeters, or between about 15 millimeters and about 50 millimeters, or between about 15 millimeters and about 45 millimeters, or between about 15 millimeters and about 40 millimeters, or between about 15 millimeters and about 35 millimeters, or between about 15 millimeters and about 30 millimeters, or between about 15 millimeters and about 25 millimeters, and other sizes suitable for ligament or tendon repair.

The scaffolds can be prepared in any desired shape. For example, the scaffolds can be prepared with straight edges or rounded edges. The scaffolds can be in the shape of a sphere, a cube, a cylinder (e.g., circular cylinder, elliptical cylinder, etc), or any polygon. In the case of a polygon, the polygon can have any of its sides straight or rounded. To the extent that a scaffold has a flat base (e.g., not a sphere), an edge can be at a right angle from the base, or at an oblique angle from the base. Edges for a scaffold need not be at the same angle, e.g., a cylindrical scaffold can have one edge (in side view) at a right angle, and the other edge (in side view) at an oblique angle; or both edges can be at different oblique angles or both right angles, etc.

In some embodiments, the scaffold is substantially cylindrical, where, in some embodiments the cylinder is a circular cylinder and in others an elliptical cylinder. In some cylindrical embodiments, the scaffold is a right angled cylinder, while in some embodiments, the scaffold is an oblique cylinder.

Compositions of the disclosure are useful for, e.g., repairing a tear or rupture of a ligament or tendon, particularly located around or within a joint, e.g., knee or shoulder joint.

### Absorption and Retention

The scaffolds described herein are engineered to maximize absorption and retention of physiological fluids, e.g., blood, synovial fluid, saline, and cell suspensions, providing an environment conducive for biological integration and tissue regeneration. This property supports not only the initial surgical workflow, where rapid fluid uptake and retention is needed for graft preconditioning and stabilization, but also the longer-term healing process, offering sustained nourishment and mechanical protection to developing tissue.

Absorption properties are controllable across a wide spectrum through the judicious selection of scaffold composition, structure, and physical characteristics. In some embodiments, scaffolds exhibit interconnected porosity, with pore volumes ranging from about 10% to greater than 80% and pore diameters from approximately 10 nm up to several hundred microns. These features enable rapid uptake of fluids by capillary action and retention through hydrostatic, adhesive, and physical entrapment mechanisms.

The clay component, particularly when modified with amino acids and mineralized in situ with, e.g., hydroxyapatite, imparts enhanced hydrophilicity, swelling capacity, and surface charge, all of which contribute to absorption kinetics. Increased polypeptide (collagen) content also correlates with higher absorption, as these proteins possess native affinity for water and blood, and provide a matrix for cellular infiltration. The presence and orientation of bioabsorbable reinforcement structures (sutures, mesh) does not appear to impede absorption, and may facilitate fluid distribution, particularly in zig-zag and spiral configurations.

Scaffolds of the disclosure typically absorb between about 35% and over 80% of their dry weight in blood, saline, or synovial-like fluid within the first few minutes of exposure, and up to 85% or more over 24 hours. Retention studies demonstrate that these fluids are held effectively within scaffold pores even after vigorous saline irrigation or mechanical manipulation, supporting durability during and after surgical intervention. Fluid absorption remained robust during the entire window of scaffold degradation, ensuring available vascular and nutrient support to infiltrating cells and regenerating tissue.

Retention is further supported by customizable degradation profiles. As scaffold material resorbs, newly integrated tissue gradually replaces the scaffold mass, and fluid-handling properties transition accordingly. Data showed that high-collagen, HAPclay-enriched scaffolds maintained both their absorption and retention capacities through multiple timepoints (up to and beyond 29 days in synovial fluid).

These absorption and retention characteristics can be further purified or tuned through variation in clay species, mineralization protocol, polymer blends, polypeptide ratios, and scaffold geometry, as well as through post-processing treatments (e.g., crosslinking, lyophilization, or surface modification).

Beyond facilitating cell and factor delivery at surgery, enhanced absorption of autologous blood or bioactive solutions supports host-specific regenerative signaling, immune compatibility, and early vascular ingrowth. The scaffold's rapid and reproducible absorption profile is thus an important component supporting biological activity, graft fixation, and overall tissue repair.

Ranges, process details, and preferred compositions discussed herein support performance equivalent to, or exceeding, that observed in the cited experimental data. Discussion and conclusions regarding clinical utility, tissue integration, and surgical workflow efficiency highlight the advancement over prior art in the field of ligament/tendon reconstruction scaffolds the embodiments of the disclosure provide.

Experimental methods, specific absorption protocols, and retention data are described in detail in the Examples section.

### Degradation

The scaffolds of the present disclosure are engineered to provide tunable and predictable degradation profiles in physiological environments, e.g., synovial fluid. Controlled degradation is important to balancing mechanical stability during early repair with eventual replacement by native tissue, thereby supporting both acute fixation and long-term biological integration.

Scaffold degradation rates are modulated by adjusting the ratios and types of structural components, e.g., clay, polymer, polypeptide, and calcium-based mineral. Higher collagen content generally leads to more rapid scaffold resorption, while increased polymer (e.g., PCL, PLGA, or PLA) concentration provides extended durability. In situ mineralized clays (e.g., HAPclay), including those modified with amino acids, contribute to structural resilience and gradual bioresorption, producing a tailored release of mineral ions that further stimulate cellular activity and matrix deposition.

The disclosure encompasses scaffolds with degradation timeframes ranging from several days to multiple months, depending on composition and intended clinical use. In some embodiments, the scaffold retains mechanical integrity for at least two weeks in synovial fluid, supporting tissue ingrowth, and then degrades steadily over the following four to twelve weeks. Specific compositions—such as those within the CO40HC10 to CO70HC12 range, varying collagen and HAPclay loadings—may achieve 20-80% mass loss after 29 days in simulated joint environments. These ranges can be adjusted via process parameters (e.g., freeze extraction, crosslinking, thermal treatment), allowing customization for particular anatomical sites or patient requirements.

Degradation is confirmed and quantified through analytical studies, including mass loss measurements, SEM imaging, and monitoring of mechanical property retention over time. The scaffold's ability to maintain function during gradual resorption ensures procedural durability and continuous support for cell migration and new tissue formation. Experimental results show that pore architecture, fiber alignment, and reinforcement pattern also influence degradation by affecting, e.g., fluid ingress and enzymatic accessibility.

Further, scaffold degradation releases functional byproducts, such as collagen peptides, mineral ions, that can stimulate host tissue regeneration and modulate inflammation. These benefits surpass the capabilities of existing materials, which typically degrade too quickly or too slowly for optimal ligament and tendon repair.

The degradation parameters disclosed herein are scalable, and equivalents are contemplated for higher or lower rates, alternative mineral phases, blended polymers, and diverse polypeptide sources. Scaffolds may be further modified to include agents (e.g., antibiotics, angiogenic factors, or stem cells) that themselves modulate degradation and tissue remodeling.

Scaffolds of the disclosure can enable an effective balance between immediate mechanical needs and sustained cellular healing. The interplay between scaffold configuration, composition, and degradation can be tailored to maximize clinical benefit.

Experimental protocols, complete methodology, and quantitative degradation data supporting these statements are provided in the Examples section.

### Mechanical Properties

The scaffolds disclosed herein are designed to provide optimized mechanical properties for ligament, tendon, and soft tissue repair, achieving a balance between initial strength, flexibility, and durability throughout degradation and tissue ingrowth.

Mechanical properties are tunable across a broad spectrum through variation in scaffold composition, reinforcement configuration, and structural design. By adjusting ratios of polymer, polypeptide, clay, and mineral components, scaffolds can be customized for clinical indications ranging from high-load ligament reconstruction to low-demand tendon augmentation. The integration and orientation of bioabsorbable reinforcement structures, such as parallel, spiral, zig-zag, and composite patterns, enable direct modulation of tensile behavior, handling, and resistance to surgical stresses.

Some embodiments exhibit a compressive modulus ranging from approximately 0.8 MPa to 3.8 MPa prior to exposure to physiological fluids, and retain at least 0.5 MPa throughout clinically relevant degradation intervals. Tensile modulus varies with reinforcement orientation and density. Zig-zag and hybrid patterns have demonstrated superior values, generally ranging from about 24 MPa to 27 MPa or more, with ultimate tensile strength sufficient for intraoperative manipulation and postoperative load bearing. These moduli can be further refined by varying reinforcement material, mesh structure, or scaffold geometry.

Mechanical performance can be further influenced by porosity, pore size distribution, and clay/mineral phase integration. Higher collagen and HAPclay content typically corresponds to increased elasticity and tunable degradation, while greater polymer blend ratios confer additional stiffness and durability. Modifying manufacturing parameters (e.g., freeze-extraction, freeze-drying, lyophilization, or 3D printing) enables additional control over scaffold texture, microarchitecture, and fiber alignment.

Properties are validated and extended by mechanical testing, including stress-strain analysis, compression, and tensile testing, performed before and after scaffold exposure to synovial fluid or simulated physiological conditions. Data confirm that the disclosed scaffolds retain necessary mechanical integrity for surgical passage, fixation, and early tissue support, while gradually transferring load to regenerating host tissue as the scaffold degrades.

The described mechanical advances are significant: the composite design provides superior handling, suture passage, and intraoperative resilience, while supporting patient-specific repair across diverse anatomical and biomechanical scenarios. These features represent a substantial improvement over conventional matrix or suture devices, which are constrained by limited modulus, rapid degradation, or inflexible design.

Mechanical property ranges, configuration options, and equivalents described herein are exemplary and scalable. Performance parameters can be tailored for higher or lower modulus, increased extensibility, or specific compatibility with autograft, allograft, or synthetic tissue, and further support both current and future claims. Experimental testing protocols and quantitative data are provided, in detail, within the Examples section.

### Scaffold Variants and Compositional Analysis

The present invention encompasses a broad spectrum of scaffold compositions and architectural variants, each tailored for mechanical, biological, and clinical performance in ligament, tendon, or soft tissue repair.

Scaffold variants are constructed using different relative concentrations of polymer, polypeptide (such as collagen or gelatin), biocompatible clay (native or amino acid-modified), and in situ mineralized phases (e.g., hydroxyapatite, other calcium phosphates). In some embodiments, the scaffold composition incorporates about 40-70% polypeptide, about 20-55% polymer, and about 5-12% mineralized clay by weight, although ranges as wide as about 1% to 80% for any individual phase are contemplated to accommodate alternative applications, patient needs, and manufacturing constraints.

Architectural variants include modifications in overall geometry (cylindrical, elliptical, oblique), pore size distribution (from tens of nanometers to several hundred microns), surface texture, edge definition, and the configuration of bioabsorbable reinforcement structures (including parallel, spiral, zig-zag, mesh, or combined patterns). The central axial through-hole can vary from about 4 mm to about 12 mm or more in diameter and about 4 mm to about 50 mm or more in length, supporting a wide range of graft dimensions, tunnel placements, and fixation techniques.

Compositional analysis confirms that scaffold performance can be modulated by shifts in component ratios, by blending natural and synthetic polymer phases, by varying the type and level of clay modification, and by adjusting mineralization protocol details (e.g., precursor concentration, pH, temperature, sequence). These factors enable the manufacture of scaffolds optimized for rapid absorption, measured degradation, desirable mechanical properties, and enhanced cell compatibility, as substantiated across the spectrum of experimental data described herein.

Comparative analysis of scaffold variants demonstrates that increased collagen content accelerates degradation and absorption, while enhanced clay mineralization bolsters mechanical stability and tissue integration over longer implantation intervals. Hybrid and alternative variants, including mixed or layered composites, custom reinforcement arrays, or multi-agent incorporation, extend the utility of the invention to complex clinical scenarios, including multi-tendon repairs, bone-ligament junctions, and regenerative medicine workflows. The ability to fine-tune scaffold variants positions the invention as a platform for innovation, adaptation, and patient-specific therapy, enabling broad patent protection and future modification.

Compositional data and full experimental details, including specific variant formulation, process conditions, and measured properties, are described elsewhere herein.

### Biological Activity

The scaffolds disclosed herein demonstrate robust biological activity, designed to promote cellular adhesion, proliferation, differentiation, vascularization, and integration of native and engineered tissues for ligament, tendon, and soft tissue repair. Biological performance is achieved through the combined action of scaffold composition, architecture, incorporated agents, and in vivo compatibility, and is tunable for a range of clinical applications.

Scaffolds of the disclosure support attachment and proliferation of mesenchymal stem cells (MSCs), tenocytes, and endothelial cells (e.g., HUVECs), among others, across a wide spectrum of cell densities and agent loadings. In preferred embodiments, the scaffold composition and porosity facilitate rapid cellular infiltration and matrix deposition, with hMSC and tenocyte seeding densities ranging, e.g., from about 104 to 10⁷ cells/cm³ readily accommodated. Surface properties, pore size distribution, and reinforcement orientation further enhance cell migration and integration, as demonstrated by live/dead imaging, histological analysis, and gene expression profiling.

Biological activity is validated by induction of lineage-specific markers and functional proteins, including upregulation of tenogenic genes (e.g., SCX, TNMD), extracellular matrix components (collagen I, CD44), and angiogenic markers (CD31, YAP, TAZ). The presence of in situ mineralized clay (e.g., HAPclay) and higher collagen ratios enhance stem cell differentiation, new tissue (neotissue) formation, and vascular network development, supporting repair of complex structures such as the anterior cruciate ligament (ACL).

The scaffolds allow for incorporation of biologically active agents, e.g., growth factors, cytokines, gene delivery vectors, antibiotics, anti-inflammatoires, patient-derived serum, and the like, via impregnation, loading, or in situ binding to scaffold phases. These agents may be present at concentrations from nanogram to milligram scale, providing sustained or controlled release that amplifies regenerative signaling, tissue integration, and immunomodulation. The modular nature of agent inclusion enables customization for specific patient needs, anatomical sites, or concurrent therapy protocols.

These biological properties are preserved and sustained throughout the scaffold degradation profile, ensuring continued support for cell survival, migration, and differentiation as native tissue replaces the resorbing matrix. As described elsewhere herein, the scaffolds can retain and deliver blood, serum, and injectable therapeutics under surgical conditions, further enhancing cell viability and tissue repair.

Comparative analysis shows that the disclosed scaffolds are superior to prior art systems in promoting biological responses essential for ligament and tendon reconstruction, including integration with autograft/allograft tissue, facilitation of vascular ingrowth, and modulation of host immune reaction. The invention is thus broadly applicable to orthopedic, sports medicine, and tissue engineering indications.

Experimental protocols, quantitative gene/protein expression data, and detailed cellular functional analysis are provided elsewhere herein.

### Surgical Methods, Workflows

The invention provides a range of surgical methods and workflows for ligament, tendon, and soft tissue repair utilizing the disclosed composite scaffolds, agent-loaded constructs, and integrated kits. These methods are adaptable across open, minimally invasive, and arthroscopic techniques, supporting clinical needs from routine repair to complex reconstruction.

In general, the surgical workflow begins with preparation and selection of the scaffold, either as provided in a kit or customized for patient and procedure. The scaffold, featuring in embodiments a central through-hole and suitable reinforcement pattern, is paired with biological agents or graft tissue (such as an iliotibial band autograft, allograft, or synthetic internal brace) as clinically indicated.

Preferred methods include anchoring the scaffold and graft within bone tunnels, anatomical insertion points, or soft tissue beds using fixation devices, sutures, or novel interface constructs. Scaffolds and grafts may be slid over pins or rods through pre-drilled tunnels, allowing precise placement and intimate interaction between the bioactive matrix and repair tissue. In minimally invasive cases, the workflow utilizes portals, cannulas, and custom instrumentation, taking advantage of scaffold shape-memory and mechanical resilience.

During surgery, the scaffold may be loaded with autologous blood, serum, or therapeutic agents via soaking, injection, or intraoperative mixing, ensuring immediate cell and factor delivery to the repair site. Cellular compatibility and agent release support regeneration, angiogenesis, and host integration as repair tissue matures.

A wide spectrum of surgical workflows is possible, including, for example, single- and double-bundle ACL reconstruction using IT band or alternate grafts; lateral extra-articular tenodesis (LET), over-the-top fixation methods; multi-ligament and complex soft tissue repair with multi-zone or multi-orientation scaffold variants; procedures in skeletally immature patients, where extra-physeal tunnels and non-invasive fixation are preferred; and hybrid or patient-specific approaches, customized using kit-supplied options and agent cartridges.

Procedural steps such as tunnel drilling, scaffold passage, pin placement, and fixation can be adjusted for tunnel geometry, graft size, and reinforcement layout. The methods support integration with existing surgical tools and visualization technologies (including 3D printed guides, imaging markers), as well as compatibility with emerging robotic workflows.

Clinical benefits include reduced operative time, scaffold-housed agent release without exogenous delivery, mechanical support throughout healing, and minimized risk of early failure. These outcomes, supported by the experimental data described herein, position the scaffolds of the disclosure for broad adoption and expandability as standards of care evolve.

Ranges for scaffold size, agent volume, fixation force, and surgeon-modifiable parameters encompass those shown in experimental work and equivalents thereof. Details for application, agent use, and fixation approaches are further described elsewhere herein.

### Kits

The disclosure encompasses kits for the storage, delivery, and clinical application of engineered scaffolds, enabling standardized and customized workflows for ligament, tendon, and soft tissue repair. Kits are designed to provide ready access to scaffold constructs, reinforcement materials, biological agents, and surgical accessories in a form suitable for rapid deployment in operative settings or preclinical research.

A typical kit can include at least one bioabsorbable scaffold as disclosed herein, optionally with pre-integrated reinforcement structures (filaments, mesh, braid) and, where required, a central through-hole dimensioned for specific graft and tendon placements. Scaffolds may be supplied in a range of sizes, compositions, and geometries, tailored to patient anatomy, repair type, or surgeon preference. Kits may further include accessory components such as surgical tools or molds for scaffold positioning, 3D-printed pins for graft passage, or occluding devices for controlled perforation.

Customization options can be integral to the kit design. In some embodiments, the kit may include pre-loaded biological agents, such as cells, growth factors, antibiotics, at controlled concentrations for immediate intraoperative use. Modular cartridges or vials allow selection and mixing of specific agents or scaffold variants, supporting patient-matched therapies and regenerative medicine protocols. Packaging may support aseptic handling, extended shelf life, and compatibility with common sterilization methods (e.g., ethylene oxide, gamma irradiation).

Scaffold kits may be delivered as bulk units for orthopedic surgical centers, as patient-specific packages for personalized medicine, or in multi-layered configurations supporting complex reconstructions (e.g., multiple tunnels, multi-ligament cases). Instructions for use, including preparation protocols, agent loading techniques, and recommended fixation procedures, are optionally supplied in printed or digital format. Kits may also include graphical guides or QR-linked instructional media for best practices and workflow integration.

Broader variants of the kit are contemplated, including kits with multiple scaffold sizes, various reinforcement patterns, or diverse agent formulations. Kits may further incorporate post-operative support materials (e.g., imaging markers, radiopaque beads, or tissue adhesives), surgical consumables, or ancillary devices compatible with minimally invasive, open, or robotic procedures.

Kits of the disclosure can enable streamlined delivery of advanced scaffold technology, supporting improved clinical efficiency, reduced operative times, and customizable regenerative outcomes.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1: Scaffold Design

Certain properties, including facilitating the regeneration process, offering structural support for a repaired ligament or tendon during repair and recovery, and controlled degradation rate of the scaffold, were defined for the design of a bioabsorbable, degradable, and mechanically strong scaffolds for ligament (e.g., ACL) or tendon repair. Four composite scaffolds varying the amount of collagen from 40%, 50%, 60%, and 70% and In-Situ HAPclay from 5% to 12% were fabricated (Table 1). The scaffold geometry was designed to require a smaller incision on the knee, leading to enhanced postoperative and improved rehabilitation outcomes.

TABLE 1
Scaffold specifications

| Name | Description |
|---|---|
| CO40HC5 | PCL + 40% collagen + 5% in-situ HAPclay |
| CO40HC7 | PCL + 40% collagen + 7% in-situ HAPclay |
| CO40HC10 | PCL + 40% collagen + 10% in-situ HAPclay |
| CO40HC12 | PCL + 40% collagen + 12% in-situ HAPclay |
| CO50HC5 | PCL + 50% collagen + 5% in-situ HAPclay |
| CO50HC7 | PCL + 50% collagen + 7% in-situ HAPclay |
| CO50HC10 | PCL + 50% collagen + 10% in-situ HAPclay |
| CO50HC12 | PCL + 50% collagen + 12% in-situ HAPclay |
| CO60HC5 | PCL + 60% collagen + 5% in-situ HAPclay |
| CO60HC7 | PCL + 60% collagen + 7% in-situ HAPclay |
| CO60HC10 | PCL + 60% collagen + 10% in-situ HAPclay |
| CO60HC12 | PCL + 60% collagen + 12% in-situ HAPclay |
| CO70HC5 | PCL + 70% collagen + 5% in-situ HAPclay |
| CO70HC7 | PCL + 70% collagen + 7% in-situ HAPclay |
| CO70HC10 | PCL + 70% collagen + 10% in-situ HAPclay |
| CO70HC12 | PCL + 70% collagen + 12% in-situ HAPclay |

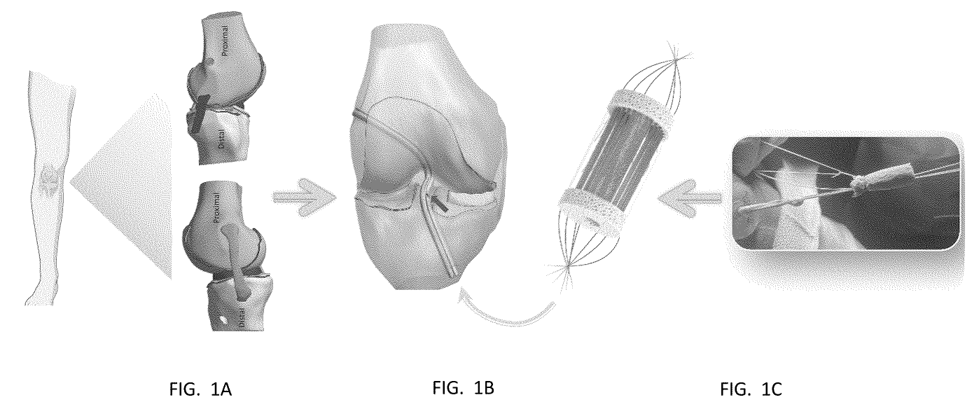

The scaffolds were reinforced with (bioabsorbable) fibers to enhance strength and facilitate better handling during surgery, e.g., ACL surgery. This enabled surgeons to manipulate the scaffolds through a cannula and tunnel before placement at the injury site. The scaffolds were designed to absorb patient blood after surgical placement, promote ligament production, and undergo gradual degradation during ligament formation. The clinical procedure for ACL repair is shown in FIG. 1.

### Example 2: Physicochemical Characterization of the Scaffolds-PBS Absorption

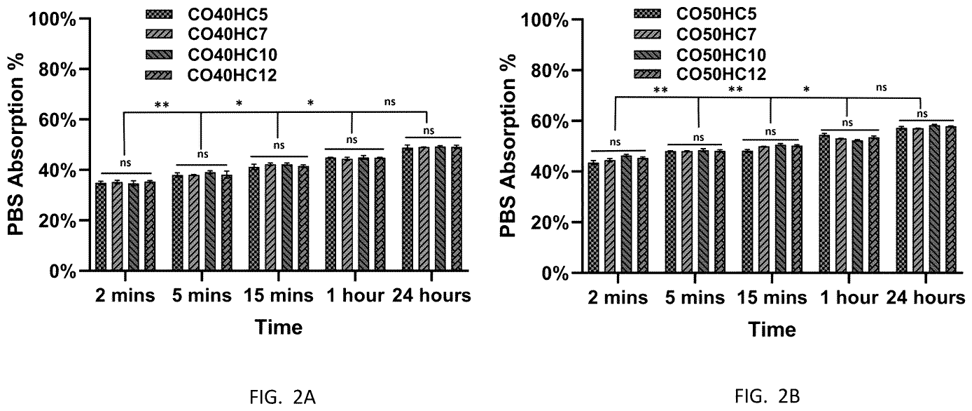

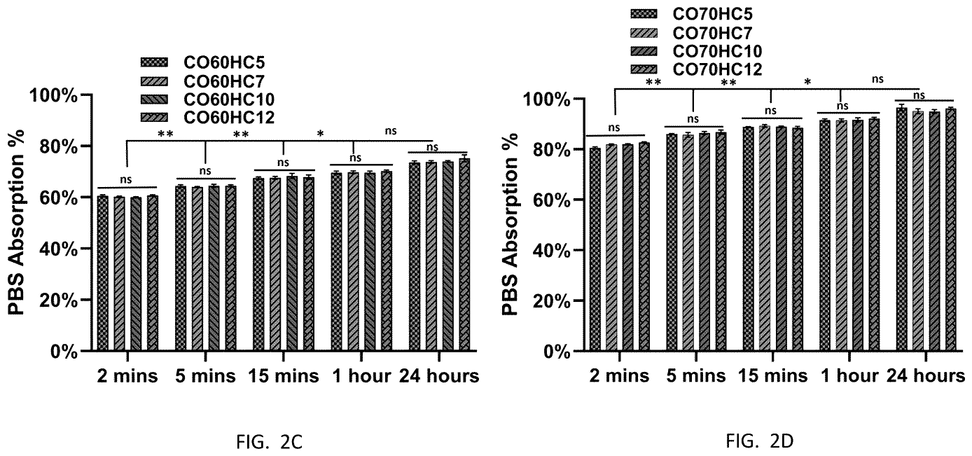

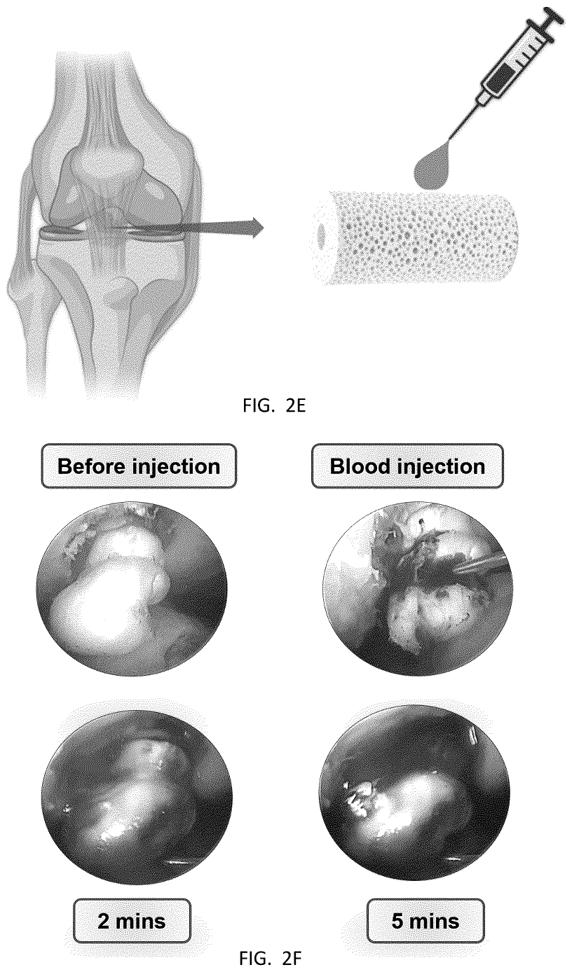

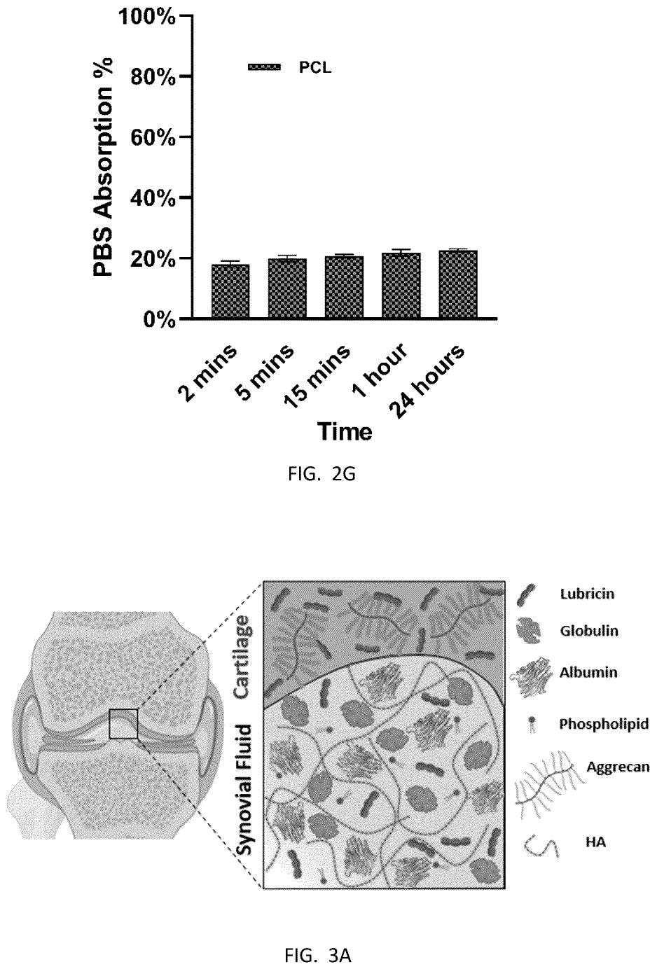

One essential property of scaffolds intended for ACL repair is their capacity to absorb patient blood during surgery, typically measured by assessing the scaffold's water or PBS absorption capacity over time, and it is referred to as the level of scaffold hydrophilicity. This characteristic reflects the scaffold's ability to absorb bodily fluids, promote the exchange of nutrients and metabolites, and support cell adhesion, growth, and differentiation. To evaluate their absorption capacity, the scaffolds were immersed in phosphate-buffered saline (PBS) for different time frames (2 minutes, 5 minutes, 15 minutes, 1 hour, and 24 hours). While scaffolds CO40HC5, CO40HC7, CO40HC10, and CO40HC12 absorbed 37% PBS within 2 minutes (FIG. 2A), those with a 60% collagen content (CO60HC5, CO60HC7, CO60HC10, and CO60HC12) absorbed 81% (FIG. 2C). Additionally, scaffolds with 70% collagen could absorb up to 85% PBS over 24 hours, as shown in FIG. 2D.

After 1 hour, the scaffolds reached their saturation point, with maximum absorption capacities of 39%, 43%, 62%, and 82% for scaffolds containing 40%, 50%, 60%, and 70% collagen, respectively. The maximum PBS absorption for pure PCL is approximately 20% over 24 hours (FIG. 2G). This is mainly due to the high hydrophobicity of PCL, which can be ascribed to the incorporation of CH₂ groups within its main structure. Collagen type I has appropriate hydrophilicity because its molecular structure includes hydrocarbon chains and hydrophilic functional groups. Following the in vitro measurement of PBS on the scaffolds, an experiment was conducted by inserting the scaffolds into the knee joint and injecting blood. The results show that while the scaffolds retained the injected blood for 2 minutes (FIG. 2E-F), by 5 minutes they had absorbed a considerable amount of patient blood, as illustrated in FIG. 2E-F.

### Example 3: Physicochemical Characterization of the Scaffolds-Synovial Fluids Degradation

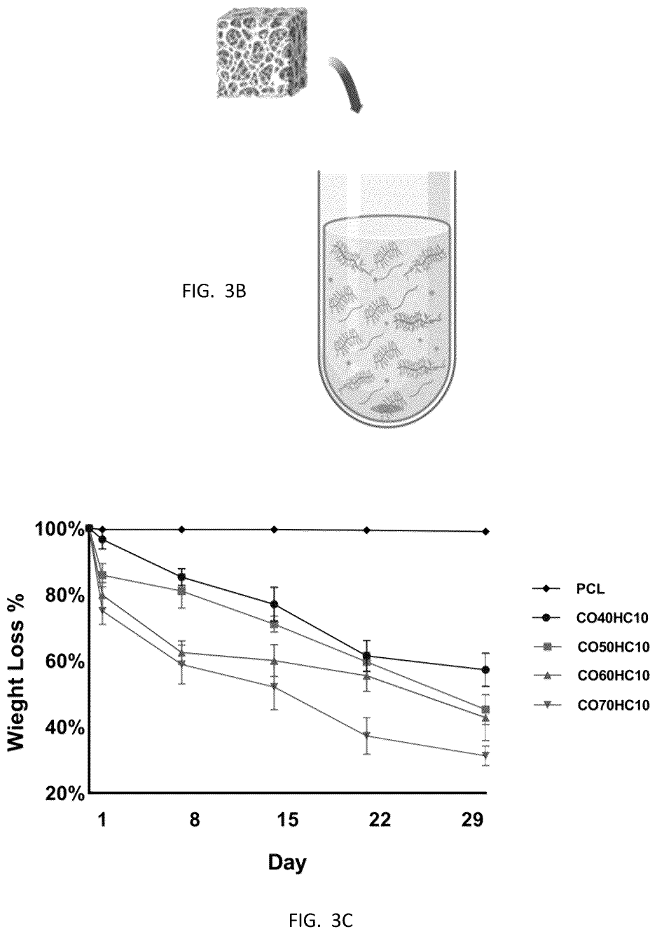

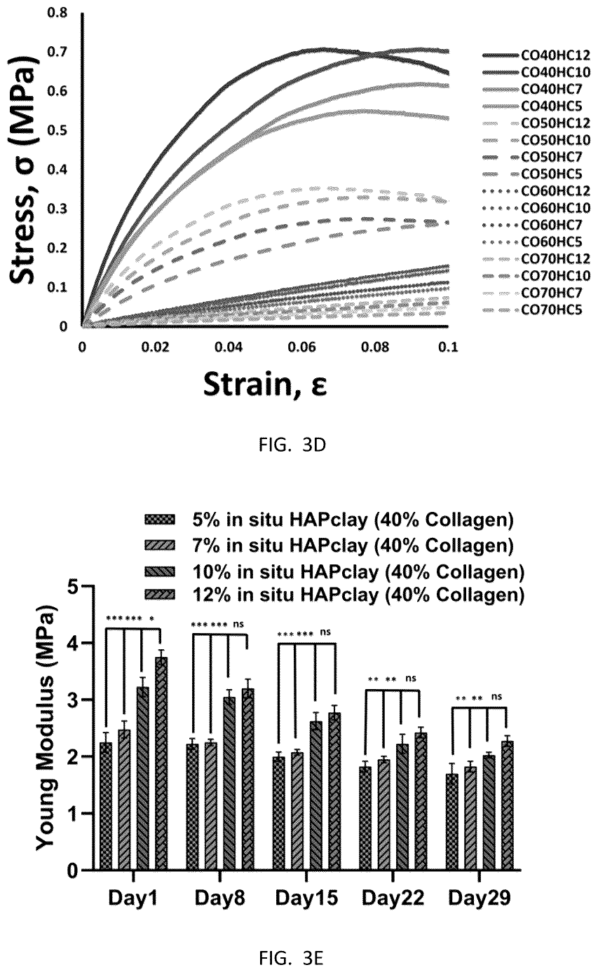

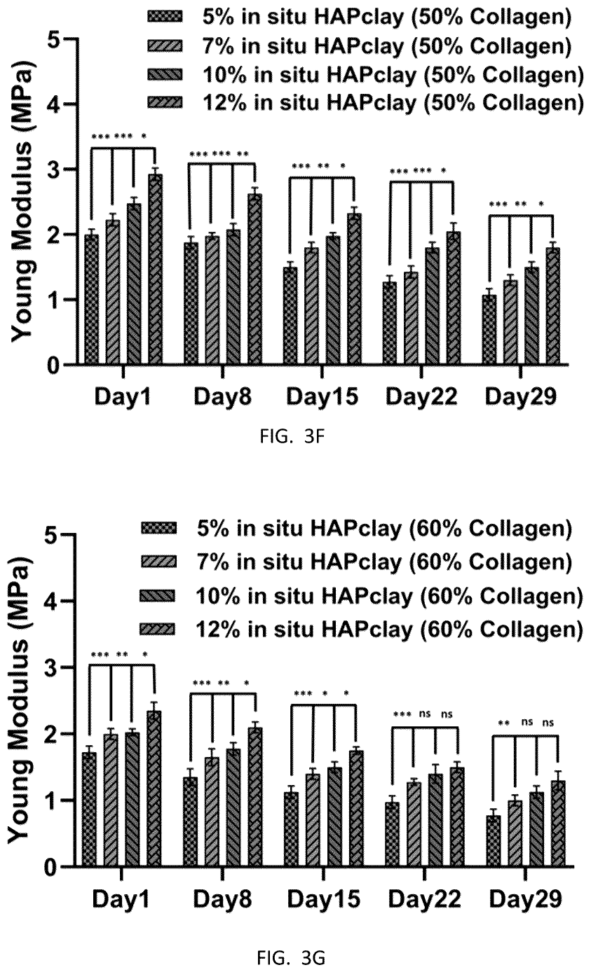

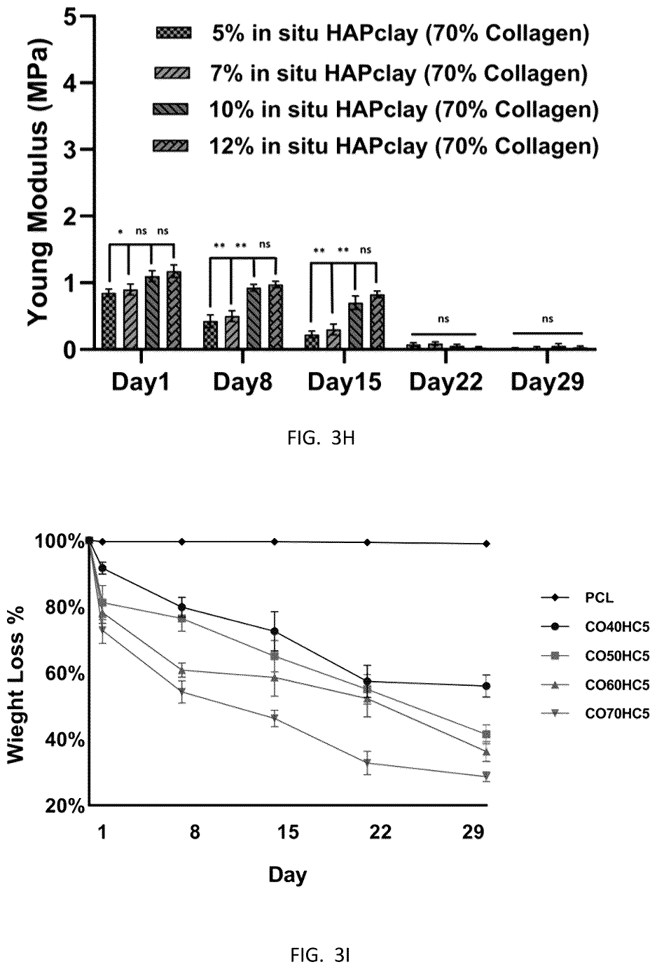

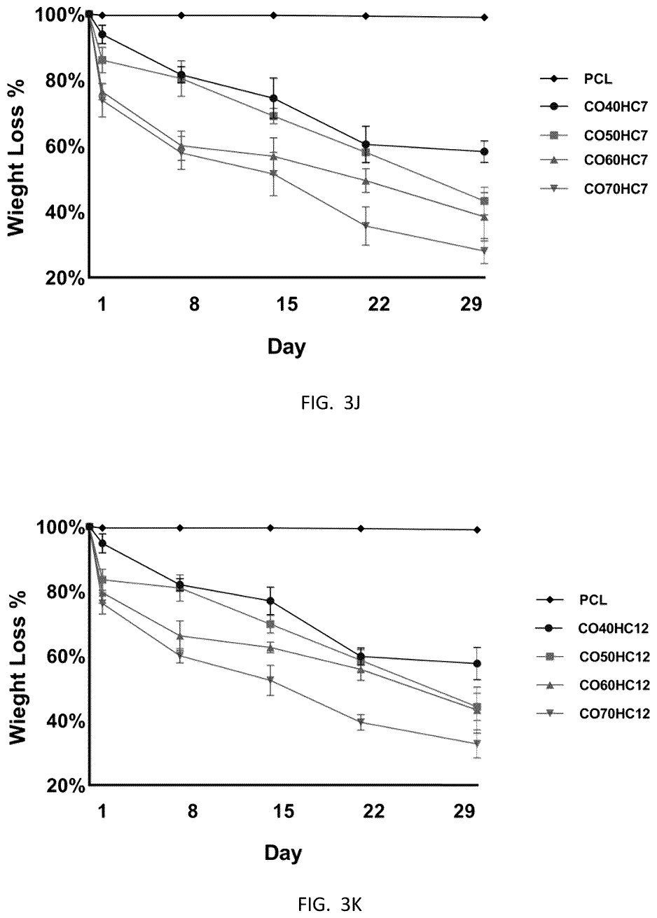

The in vitro degradation experiments utilized separate scaffolds for each group at different time points. The synovial fluids were replaced at each measurement point, and the pH levels were consistently tracked during the entire experiment. FIG. 3 illustrates the degradation percentages of the samples over 29 days in synovial fluid conditions. It's reported that the collagen could be easily degraded. In contrast, the degradation time of PCL would be much longer. When polymers come into contact with the surrounding fluids, they degrade through chain scission, resulting in low molecular weight species, monomers, and oligomers. Every biodegradable polymer has hydrolyzable bonds, making them susceptible to biochemical degradation by enzyme-catalyzed or simple hydrolysis. Unlike natural polymers, synthetic polymers are less sensitive to enzymatic hydrolysis and tend to degrade through simple hydrolysis, generating negligible to moderate foreign body reactions because their hydrolytic degradation products (lactic and glycolic acids) are frequently present in the human body's metabolic pathways. This study used a pure PCL scaffold as a control to assess the impact of in situ HAPclay and collagen type I on the degradation process. The PCL/Collagen/in situ HAPclay scaffolds showed weight loss within the first seven days, followed by a gradual degradation over 29 days. In contrast to the composite scaffolds, the pure PCL scaffolds exhibited slower degradation. These findings highlighted the significant role of collagen type I in accelerating scaffold degradation. Moreover, the results indicate that the degradation of the CO70CL10 scaffolds (66%) was significantly higher (p<0.05) compared to the CO40CL10 scaffolds on day 29 (FIG. 3C). This suggests that increasing the collagen percentage leads to a higher degradation rate of the scaffolds in synovial conditions. The degradation behavior of scaffolds with 5%, 7%, and 12% in situ HAPclay has been represented in FIG. 3I-K).

### Example 4: Physicochemical Characterization of the Scaffolds-Mechanical Properties of the Scaffolds

The microstructure, porosity, and nano-level additives have a significant impact on the mechanical characteristics of materials. The deformation behavior of nanocomposite scaffolds, composed of PCL/Collagen/in situ HAPclay scaffolds under compressive loading, is depicted in the stress-strain curves illustrated in FIG. 3D. Based on the information shown in FIG. 3D, each curve exhibited an initial linear stress region during compression that increased proportionally to the strain. This was followed by a nonlinear increase in stress with strain, indicating densification. The scaffold displayed an elastic response within the strain range of 0-0.01, representing its initial elastic portion. Subsequently, as compression continued, the scaffold's internal structure began to collapse, resulting in the compaction of the porous scaffold's microstructure. Consequently, the compressive stress in the densification region increased nonlinearly as the strain rose. FIG. 3D illustrates that the scaffold consisting of CO40HC12 exhibited the highest compressive strength among all the tested scaffolds.

TABLE 2
The Young modulus value of the scaffolds
before the degradation study

| Samples | Young Modulus (MPa) |
|---|---|
| CO40HC5 | 2.2 ± 0.28 |
| CO40HC7 | 2.4 ± 0.14 |
| CO40HC10 | 3.3 ± 0.63 |
| CO40HC12 | 3.8 ± 0.41 |
| CO50HC5 | 1.9 ± 0.52 |
| CO50HC7 | 1.8 ± 0.57 |
| CO50HC10 | 2.3 ± 0.26 |
| CO50HC12 | 2.9 ± 0.38 |
| CO60HC5 | 1.8 ± 0.28 |
| CO60HC7 | 2.1 ± 0.14 |
| CO60HC10 | 2.2 ± 0.63 |
| CO60HC12 | 2.3 ± 0.41 |
| CO70HC5 | 0.8 ± 0.15 |
| CO70HC7 | 0.9 ± 0.26 |
| CO70HC10 | 1.2 ± 0.33 |
| CO70HC12 | 1.3 ± 0.12 |

The degraded wet scaffolds were tested at intervals to measure their compressive strength throughout the degradation study. The compressive Young modulus of control data from this test are presented in Table 2. The data suggests that in all sets of the scaffolds, the Young's modulus of scaffolds made with 12% percentages of the in situ HAPclay is most influenced by incorporating the nanoclay in PCL polymer, resulting in higher stiffness. These results confirm that the interactions between in situ HAPclay and PCL play a crucial role in determining the mechanical properties of the scaffold system. Incorporating in situ HAPclay significantly impacts the overall performance and behavior of the composite, underscoring the importance of their interplay in shaping the mechanical properties of the scaffold. Moreover, increasing the amount of collagen type I reduces the Young's modulus. The scaffold containing 70% collagen demonstrates the lowest compressive strength under similar compression levels. Table 2 provides the compressive modulus values for each type of scaffold. The scaffold composed of PCL/Collagen with only 5% in situ HAPclay demonstrates the lowest Young modulus under similar compression levels in each series of samples, including PCL and 40%, 50%, 60%, and 70% of collagen: CO40HC5: 2.2±0.28, CO50HC5: 1.9±0.52, CO60HC5: 1.8±0.28, CO70HC5: 0.8±0.15, whereas 12% shows the highest Young's modulus value including CO40HC12: 3.8±0.41, CO50HC12: 2.9±0.38, CO60HC12: 2.3±0.41, CO70HC12: 1.3±0.12. The Young's modulus of scaffolds made with 7% and 10% percentages of in situ HAPclay is influenced by the lower incorporation of in situ HAPclay in the PCL polymer and collagen, resulting in an intermediate value.

Collagen percentages significantly influence the mechanical properties of scaffolds. Scaffolds containing 70% type I collagen demonstrated a 340% lower elastic modulus compared to those containing 40%. In the next step, the compression strength of the scaffolds was measured after days 1, 8, 15, 22, and 29 in the synovial fluid condition. On day 29, the CO70HC12 scaffolds nearly lost their mechanical strength, while the CO40HC12 scaffolds maintained Young's modulus of 2.3±0.41 MPa. Meanwhile, the CO60HC10 scaffolds exhibited a compressive modulus of 1.2±0.23 MPa on day 29 under synovial fluid conditions. The CO60HC10 scaffolds demonstrated the ability to retain intermediate strength over 29 days, while also showing a capacity to absorb patient blood within an acceptable range, as previously observed. This allows for improvement in the strength of the construct for ACL repair. Maintaining the mechanical properties of the scaffolds is crucial for facilitating the exchange of nutrients and growth factors, which can accelerate the healing process of ligaments. This characteristic is especially beneficial for ACL repair supported by an IT-band graft, where the scaffold must degrade more rapidly than those used in ACL replacements. This highlights the potential of the CO60HC10 scaffold, which can meet the degradation and absorption rate criteria of ACL repair scaffolds. Adjusting the collagen content and in situ HAPclay can optimize scaffold properties for specific surgical needs.

### Example 5: Physicochemical Characterization of the Scaffolds-Microstructural Characterization

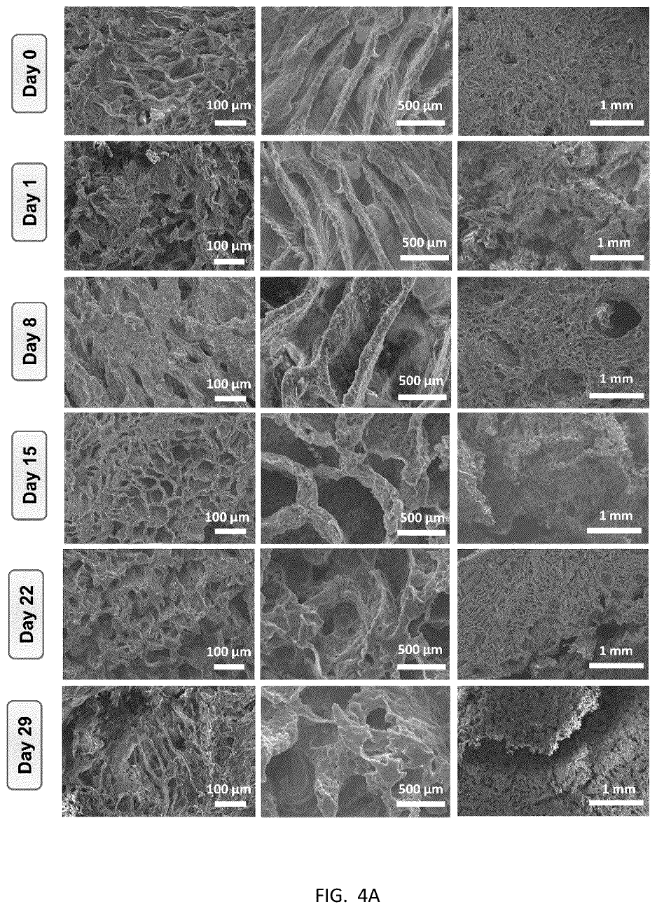

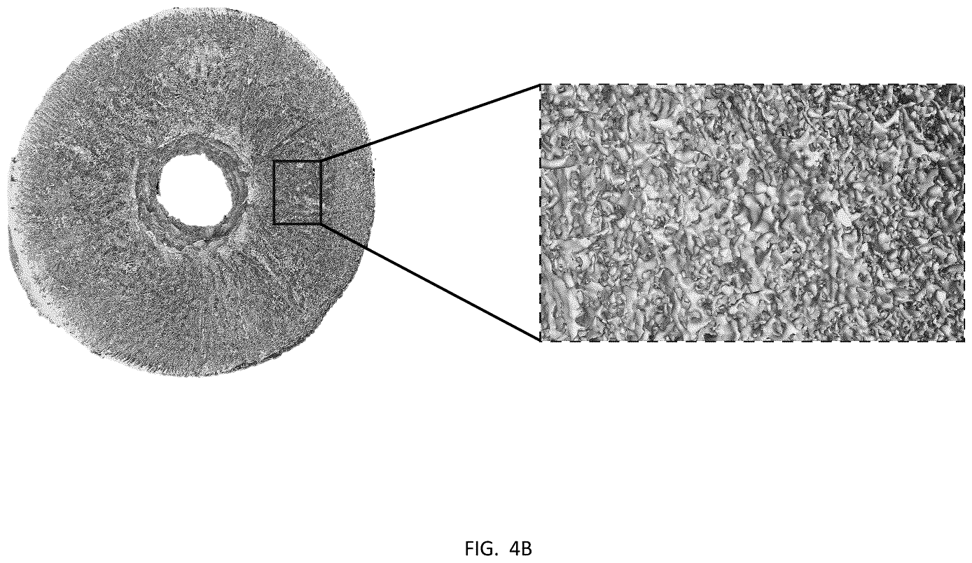

The SEM images taken both before and after degradation demonstrate the microstructural characteristics of the CO60HC10 scaffold at different magnifications. Initially, the scaffold exhibits a dense, interconnected porous network (FIG. 4B)), which is crucial for cellular infiltration and nutrient transport. At higher magnification, the images show minor surface erosion and the formation of small voids, primarily due to the degradation of the collagen component. The PCL matrix remains largely intact, maintaining the scaffold's overall structure (FIG. 4A). As the material undergoes degradation over time, subsequent SEM images show progressive morphological changes, indicative of the material's response to the synovial fluid. Over time, the images depict increased porosity and the formation of voids, while PCL remains intact for a longer period, thereby influencing the overall mechanical stability of the scaffold. Moreover, minor surface erosion and a gradual breakdown of the PCL component have been observed, which is expected due to its relatively slower degradation rate compared to collagen. These changes suggest a controlled degradation process, where the scaffold gradually loses its collagen content while maintaining mechanical stability through the PCL and in situ HAPclay components.

On Day 29, the SEM images revealed extensive degradation of scaffolds, characterized by a collapsed and fragmented structure. The scaffolds showed severe degradation, large, irregular pores, and significant material loss. The progressive loss of the collagen network, accompanied by the persistent presence of PCL and in situ HAPclay, points to a favorable degradation profile that could support ACL regeneration over an extended period.

### Example 6: Physicochemical Characterization of the Scaffolds-Differential Scanning Calorimetry (DSC)

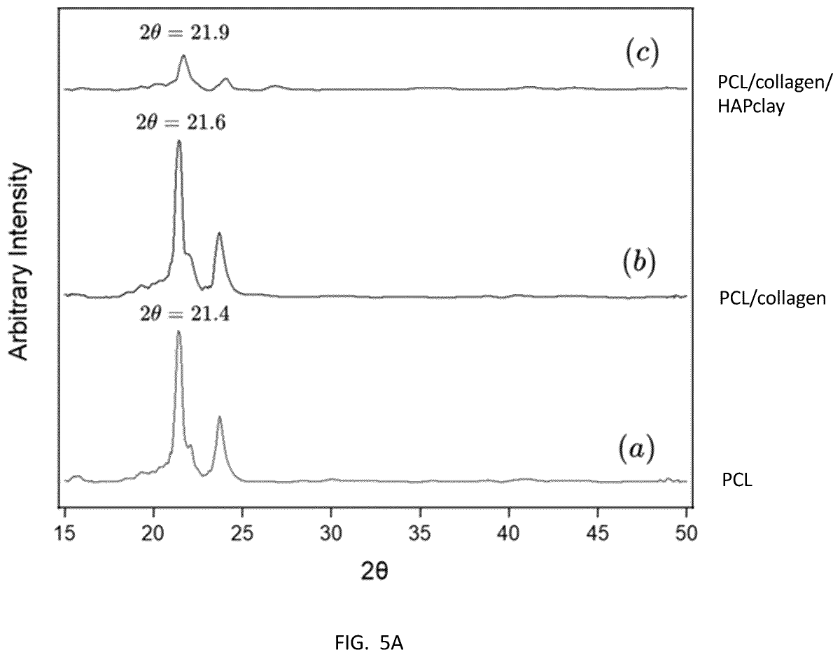

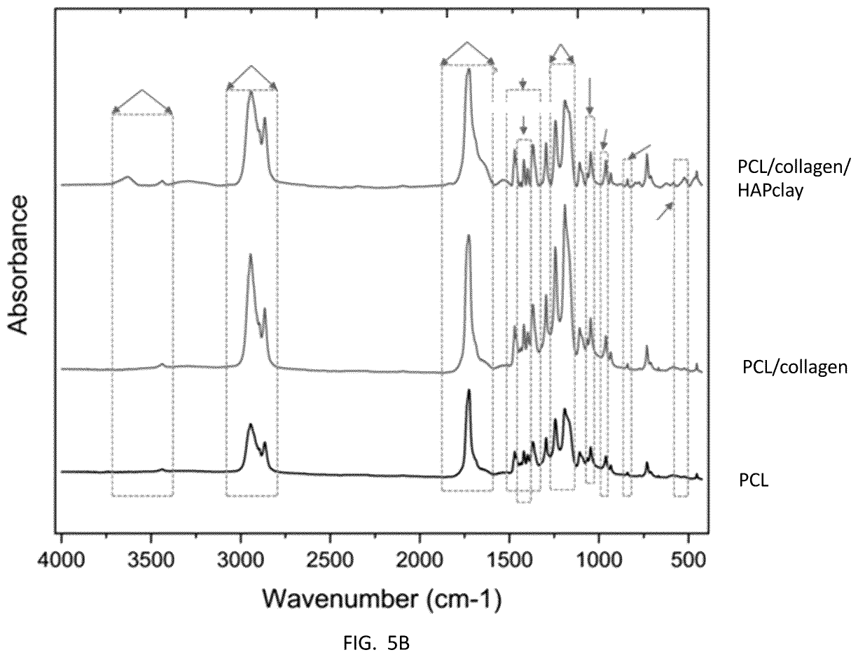

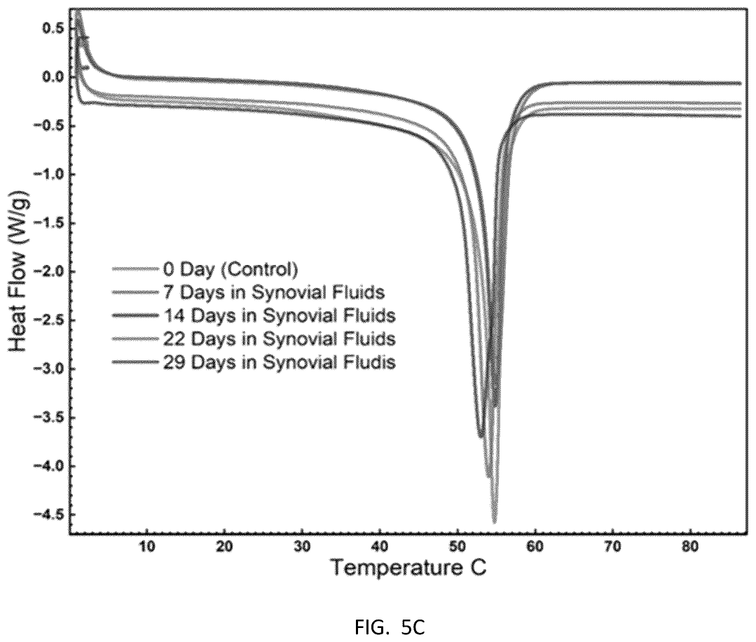

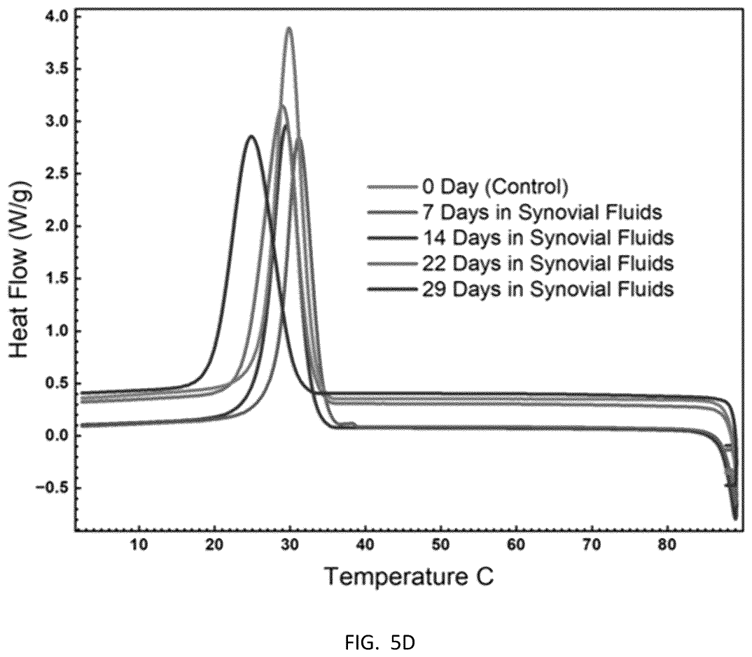

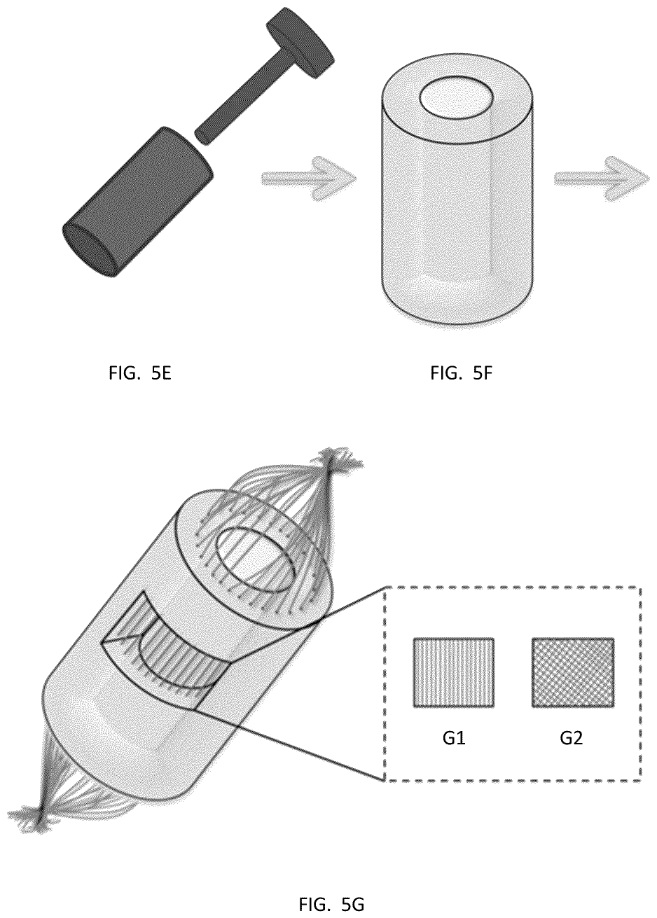

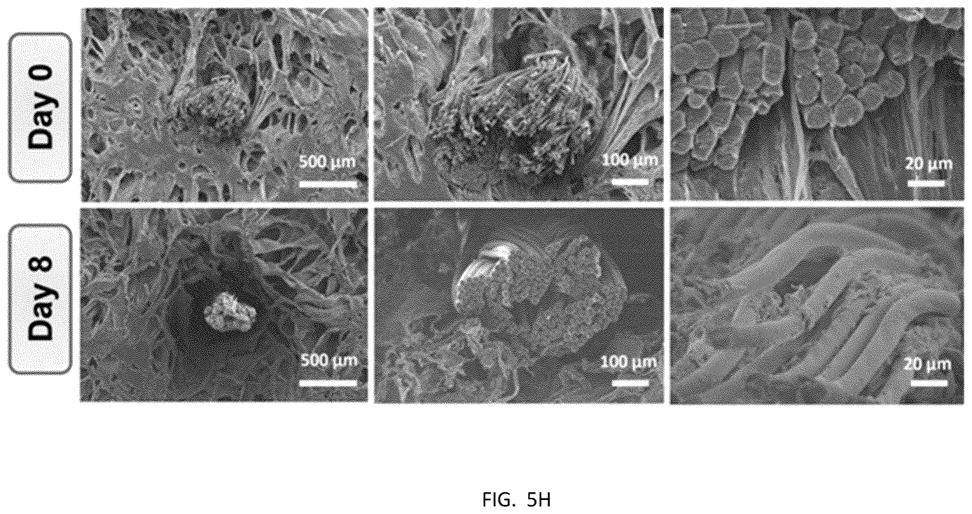

The primary limitation of PCL is its slow degradation rate, which exceeds 24 months. This slow degradation is due to its high crystallinity (approximately 45%) and hydrophobic nature. Although a lack of crystallinity is often linked to a faster degradation rate, it also results in decreased rigidity and strength. Additionally, maintaining PCL's crystallinity is crucial for its shape memory behavior. A highly desirable strategy for modifying PCL would involve accelerating its degradation rate while enhancing its mechanical properties and preserving its shape memory behavior. To tune the degradation rates, combining two or more polyesters as copolymers or blends is standard practice. Moreover, increasing the amount of collagen in the scaffold system will increase the surface area to volume ratio, consequently providing a more significant porosity to facilitate rapid degradation. FIG. 5C-D show that the endothermic and exothermic peaks decrease over time for CO60HC10 scaffolds, which is the combined effect of the degradation of the CO60HC10 scaffolds in synovial fluids. The reduction in both endothermic and exothermic peak intensities over time implies that the scaffolds undergo structural and thermal changes due to immersion in synovial fluid. These changes include decreased crystallinity, partial degradation, and potential interactions between the scaffold's components and the fluid environment. In the control sample (0 days), a prominent endothermic peak is observed around 54.8° C., corresponding to the melting temperature of PCL (FIG. 5C). As the material is exposed to synovial fluid, a shift in the melting peak occurs, with slight broadening evident as early as 7 days. This initial shift indicates a minor thermal response, suggesting that the synovial fluid begins interacting with the composite but does not significantly affect its crystallinity or melting behavior within the first week. With extended immersion times of 14, 22, and 29 days, more changes occur in the thermal properties of the composite. The broadening of the endothermic peak and the shift in melting temperature suggest a decrease in PCL crystallinity, likely due to fluid absorption by the collagen matrix. By 29 days, the broadening and shifting of the melting peak become more evident, indicating that prolonged exposure to synovial fluid results in structural changes within the composite. In the cooling cycle (FIG. 5D), a sharp exothermic peak appears at approximately 33° C., indicating the crystallization of the control sample (0 days), which is likely related to the collagen component. After 7 days of immersion in synovial fluids, the exothermic peak shifts slightly and broadens, suggesting that the synovial fluid has begun interacting with the composite, influencing its crystallization behavior. By day 14, the peak narrows and shifts downward, which may indicate changes in the collagen network resulting from partial fluid absorption or structural rearrangements. More changes are observed as immersion continues for 22 and 29 days. The exothermic peak shifts further, especially after 29 days, indicating significant alterations in material structure, likely due to prolonged fluid exposure.

### Example 7: Physicochemical Characterization of the Scaffolds-XRD Analysis

The X-ray diffraction (XRD) patterns of PCL (a) PCL/Collagen (b) and PCL/Collagen/in situ HAPclay (c) highlight the evolution of crystalline and amorphous phases with changes in composition (FIG. 5A). PCL shows sharp and intense peaks at 2θ=21.4 and a secondary peak at approximately 2θ=23.8. These peaks are characteristic of PCL's semi-crystalline nature, indicating a dominant crystalline structure consistent with its known properties. PCL with collagen exhibits a similar diffraction pattern with a peak at 2θ=21.6. However, the slight shift and reduced intensity of the primary peak at 2θ=21.6 suggest partial disruption of PCL's crystalline structure due to collagen incorporation. The observed changes imply an increased amorphous character, reflecting the compatibility of PCL with collagen and the potential improvement in biological properties. The XRD pattern reveals additional features for a composite of PCL, collagen, and in situ HAPclay. The characteristic PCL peaks are present but shifted to 2θ=21.9 with reduced intensity. New peaks emerge around 2θ=25, corresponding to the crystalline in situ HAPclay phase. After interplanar spacings (d) calculation from the XRD patterns, the d-spacing values of 4.15 Å and 3.74 Å correspond to the crystalline planes of pure PCL, consistent with its semi-crystalline nature. PCL incorporating collagen shows slight reductions in d-spacing to 4.11 Å, indicating a subtle disruption of the PCL crystalline structure by the amorphous collagen matrix. PCL, collagen, and in situ HAPclay exhibit a broader range of d-spacing values, with 4.06 Å and 3.56 Å attributed to PCL and a distinct 2.79 Å peak characteristic of the hydroxyapatite phase. These results confirm the effective integration of in situ HAPclay into the composite, enhancing its structural and bioactive properties.

### Example 8: Physicochemical Characterization of the Scaffolds-FTIR Spectroscopy

The FTIR results for CO60HC10 within the 500-4000 cm-1 range are shown in FIG. 5 B. The spectrum of PCL presents a set of typical strong carbonyl group absorption bands at 1726 cm-1 (C═O, stretching), 2866 cm-1, and 2949 cm-1 (CH2, asymmetric stretching). Characteristic peaks at 1270 and 1185 cm-1 corresponded to the C═O bond in PCL. While the PCL/in situ HAPclay spectra retain the primary characteristic bands of PCL, additional bands appear, confirming the incorporation of in situ HAPclay. The P—O bending vibration from the phosphate groups in situ HAP typically appears in this range, particularly around 550-600 cm⁻¹. The newly observed bands at 604 and 568 cm⁻¹ correspond to PCL/in situ HAPclay and PCL/Collagen/in situ HAPclay. Additionally, MMT clay modified with amino valeric acid exhibited bands at 1716, 1710, and 1724 cm⁻¹, confirming the inclusion of amino acids in the modified clays. Si—O stretching bands were observed at 1031 cm-1 for MMT clay and amino valeric acid-modified MMT clay. The bands detected at 958, 914, 912, and 918 cm⁻¹ are assigned to Al—OH deformation, while those observed at 798, 794, 794, and 796 cm⁻¹ correspond to Al—FeOH deformation. In addition to these bands, the CO60HC10 scaffold, which contains PCL, collagen, and in situ HAPclay, displays absorption bands associated with collagen. The Amide I and Amide II peaks are observed at 1659 cm⁻¹ and 1555 cm⁻¹, respectively. Moreover, the band at 3635 cm-1 is assigned to the O—H stretching vibrations, primarily from the structure's hydroxyl groups (O—H).

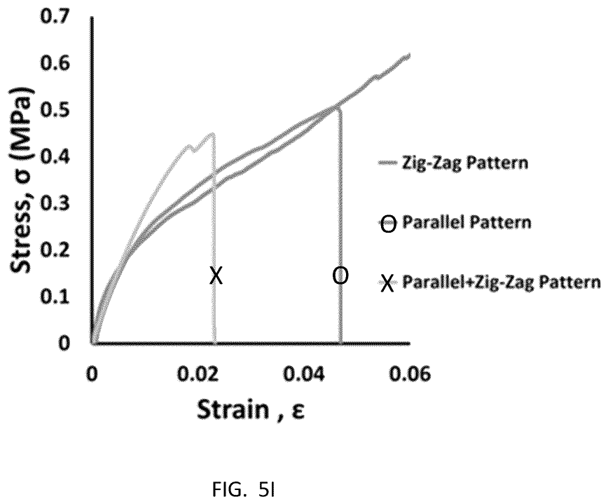

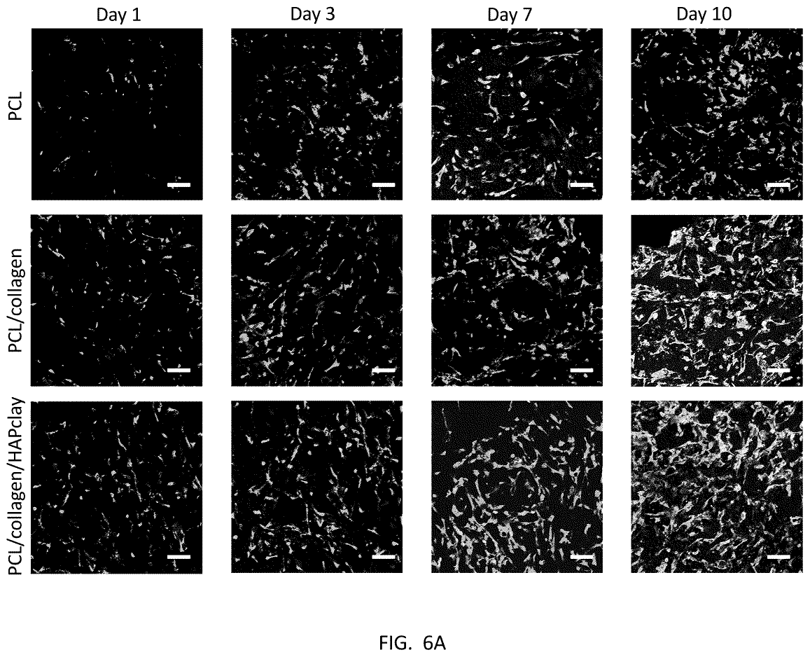

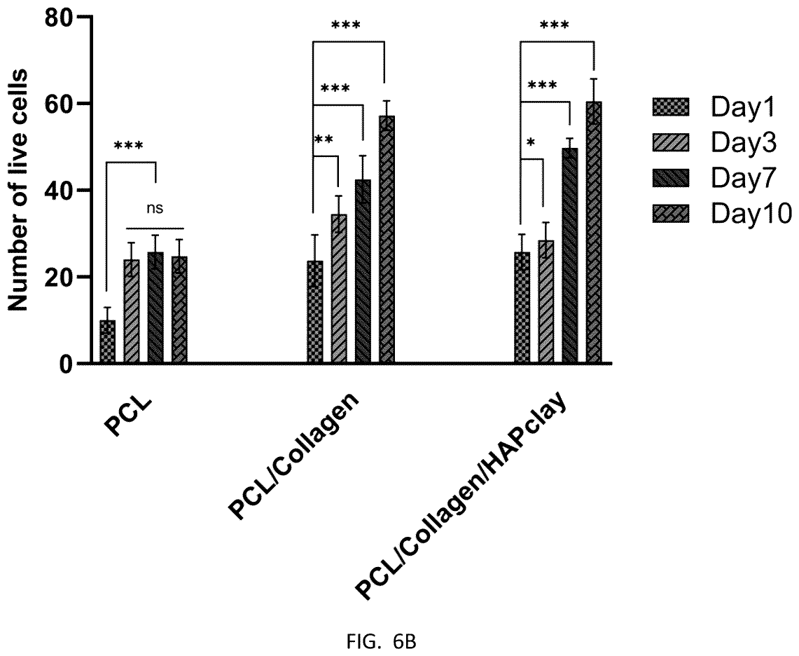

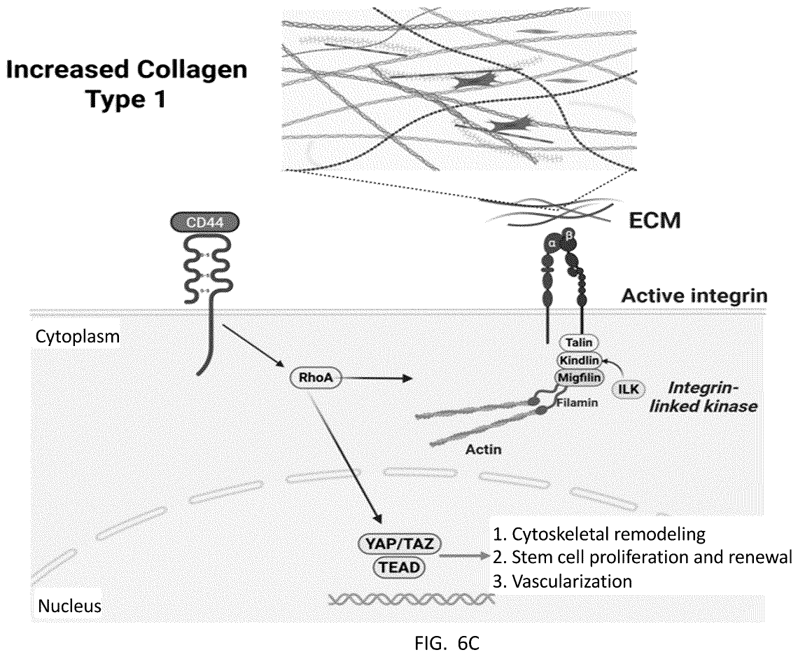

### Example 9: Physicochemical Characterization of the Scaffolds-Scaffolds Reinforcement

[RENUMBER?] A pin (here, 6.5 mm; FIG. 6, panel (a)) was fabricated using 3D printing (SLM) to create a hole in the scaffold (FIG. 6, panel (b)), facilitating the surgeon's arthroscopic application.

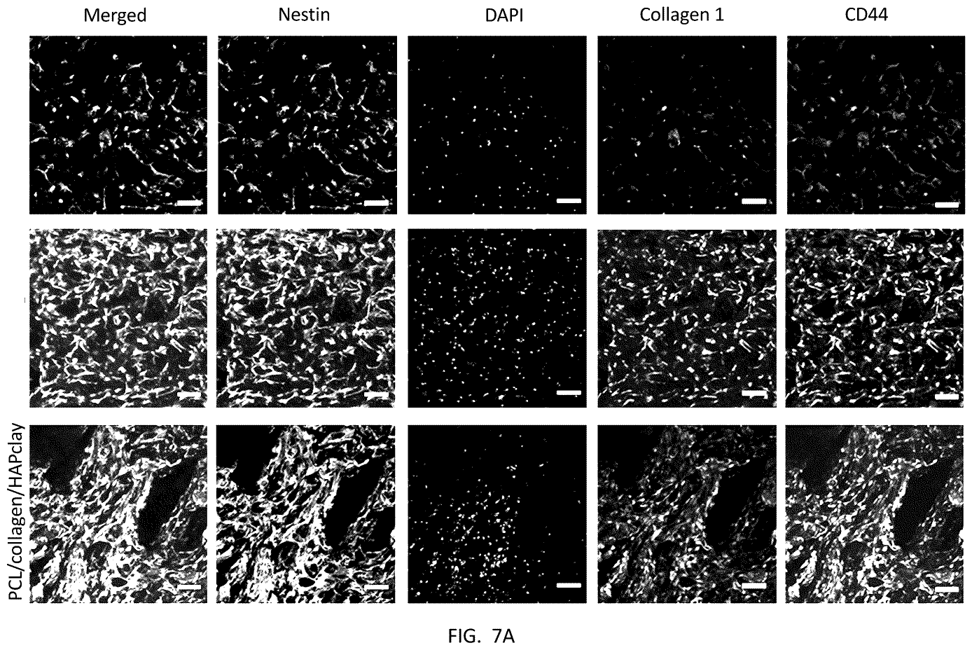

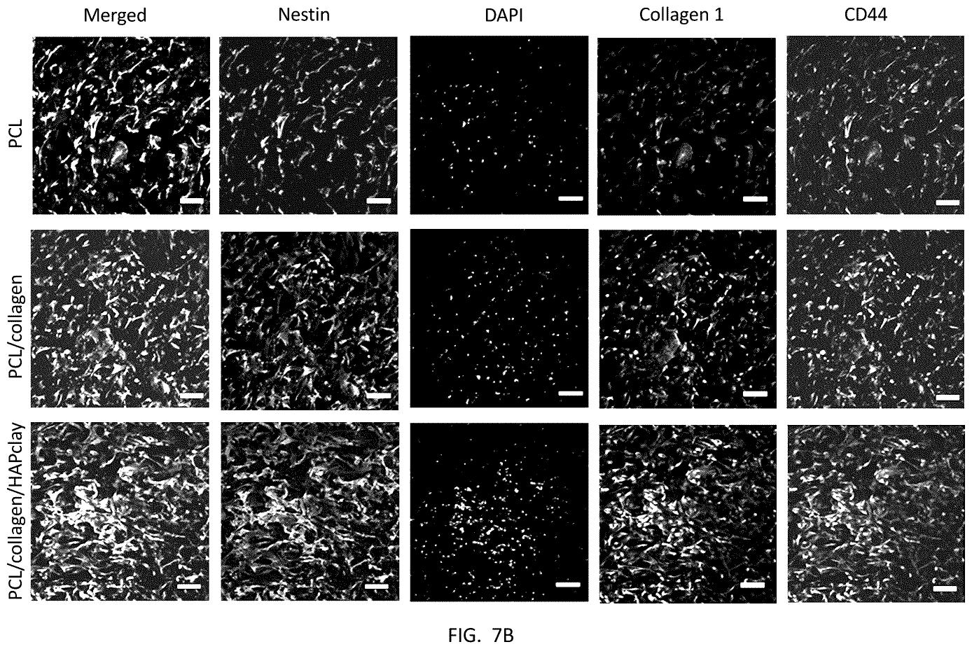

Fibers with different orientations were incorporated into the scaffolds to improve scaffold mechanical properties, e.g., tensile properties, allowing a surgeon to, e.g. for certain ACL procedures, pull the scaffolds through the tunnel of the femur and tibia in the knee joint. Fibers have been incorporated in three different orientations: one orientation follows a parallel design (FIG. 6, panel (c)), where the fibers are aligned parallel to the center point of the scaffold, and another utilizes a spiral and zig-zag pattern (FIG. 6, panel (d)), creating an interwoven structure. The third design is a combination of the previous orientation (FIG. 6, panel (e)). SEM images of fiber incorporation can be seen in FIG. 7.

### Example 10: Suture Mechanical Properties and Microstructure

The scaffolds with embedded sutures exhibited varied behavior during the tensile test. The tensile mechanical properties of the CO60HC10 scaffolds (clinically finalized by an orthopedic surgeon) with sutures are represented through stress-strain curves (FIG. 5I). The zig-zag sutures design demonstrated a relatively higher Young's modulus, with values of 26.77±0.12 MPa, compared to the parallel design and the combination of parallel/Zig-Zag designs, which had values of 24.43±0.16 MPa and 25.27±0.35 MPa, respectively (Table. 3). The Zig-Zag design allows for more strain and flexibility, preferably from a surgeon's perspective, when handling during surgery. In terms of UTS values, the zig-zag pattern achieved the highest stress before failure, 0.62±0.28 MPa, while the parallel and combined patterns showed comparatively lower UTS values (0.50±0.17 MPa and 0.48±0.31 MPa, respectively). Overall, all designs possess sufficient mechanical strength to be pulled through the femoral and tibial tunnel and positioned at the injury site. These orientations are designed to replicate the anisotropic properties of native tissues, providing the mechanical strength and flexibility essential for tissue regeneration.

FIG. 5H focuses on the degradation behavior of the CO60HC10 scaffold with sutures over time. On Day 0, the sutures appear well-integrated with the scaffold matrix, maintaining a smooth and intact morphology. The sutures are firmly embedded, ensuring stability and mechanical reinforcement. By Day 8, degradation is observed around the suture region, with clear evidence of surface erosion. These images reveal a partial disintegration of the suture material, suggesting interaction with the surrounding environment.

TABLE 3
The Young modulus value of the scaffolds
with different pattern of sutures

| Sutures Patterns | Young Modulus (MPa) |
|---|---|
| Zig-Zag Pattern | 26.77 ± 0.12 |
| Parallel Pattern | 24.43 ± 0.16 |
| Zig-Zag and Parallel Pattern | 25.27 ± 0.35 |

### Example 11: PCL/Collagen/In Situ HAPclay Scaffolds Provided a More Suitable Environment for Long-Term Growth

In FIG. 6, the live-dead imaging indicated that hMSCs could grow up to 10 days in all three scaffold systems. However, we observed that PCL scaffolds experience less growth of hMSCs over time compared to the other two scaffold types. On day 1, hMSCs were attached more to the PCL/Collagen and PCL/Collagen/in situ HAPclay (CO60HC10) scaffolds than to the PCL scaffolds. No significant difference was observed on day 3 between all three scaffolds. However, on days 7 and 10, the PCL/Collagen/in situ HAPclay (CO60HC10) scaffold had more cell growth than the others. To further quantify the growth of the hMSCs on each scaffold, the number of live cell aggregates per 650×650 μm area was counted at each time interval, FIG. 6B. The average live cell count was highest on day 10 for the PCL/Collagen and PCL/Collagen/in situ HAPclay (CO60HC10) scaffolds without suture, which were significantly higher than those of the PCL scaffolds. Additionally, a significant increase in cell growth was observed on day 7 for the PCL/Collagen and PCL/Collagen/in situ HAPclay (CO60HC10) scaffolds. The average cell count for PCL/Collagen and PCL/Collagen/in situ HAPclay (CO60HC10) with suture has also been depicted in FIG. 6D (Figure S6), and there was no significant difference observed between scaffolds with and without sutures.

### Example 12: Biological Response-Increased Collagen Production in PCL/Collagen/In Situ HAPclay Scaffolds with and without Sutures

The live-dead images were substantiated with immunofluorescence images in FIG. 7A and FIG. 7B, revealing a significant finding. There was no discernible difference in cell growth and nestin expression from scaffolds with and without sutures. The hMSCs on day 10 should not restrict cellular activity in the presence of sutures. Additionally, confirming the live-dead data, PCL/Collagen/in situ HAPclay (CO60HC10) scaffolds contained more hMSCs, as indicated by DAPI and nestin expression. Both PCL/Collagen and PCL/Collagen/in situ HAPclay (CO60HC10) scaffolds had increased collagen type I and CD44 expression compared to PCL scaffolds. Specifically, hMSCs on PCL/Collagen/in situ HAPclay (CO60HC10) scaffolds experienced the most collagen type I and CD44 activation. This finding highlights the significance of scaffold composition in stimulating cell activity and protein expression.

### Example 13: Biological Response-Vasculature and Growth in Co-Culture of HUVECs and hMSCs is Significantly Intense in Collagen Scaffolds

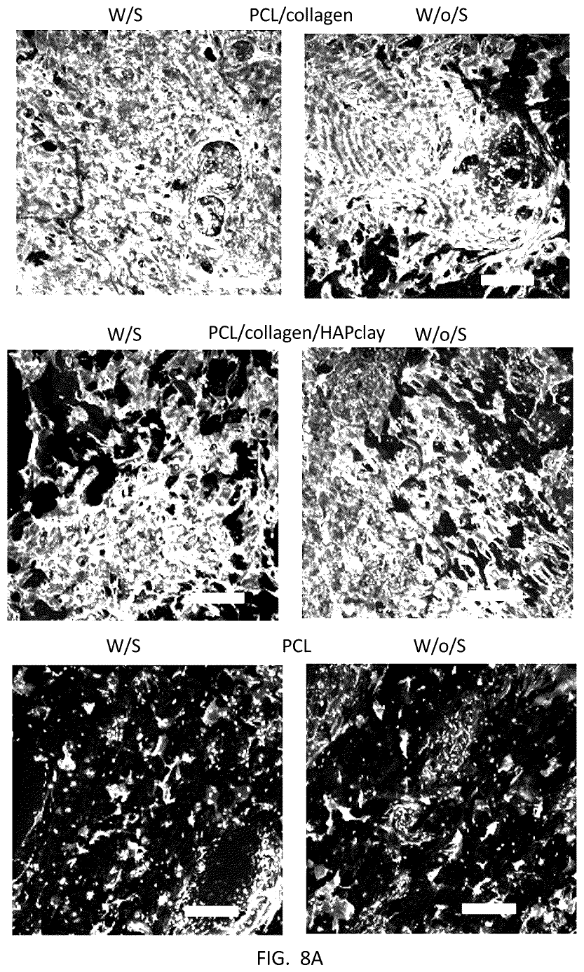

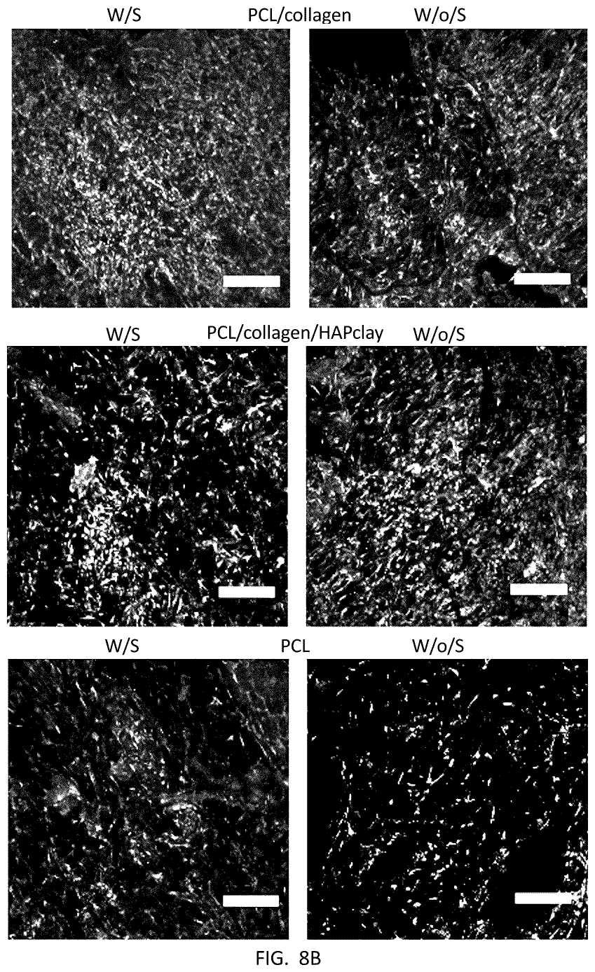

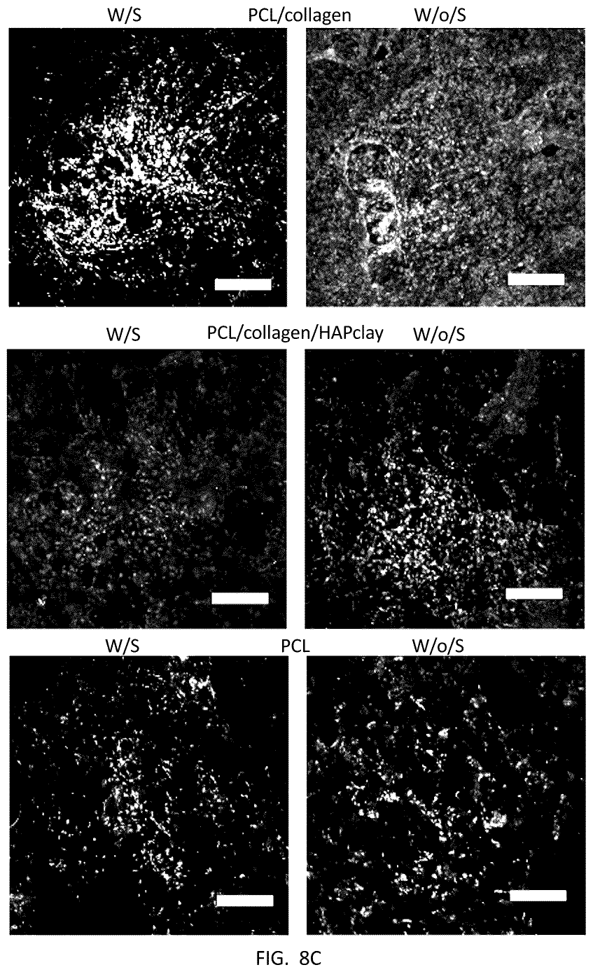

HUVECs were seeded on 10-day seeded hMSC scaffolds for 5 hours before imaging. In FIG. 8A, all samples were stained with CD31, a marker for vascular differentiation (specific to endothelial cells). We observed that the HUVECS were significantly adherent to the PCL/Collagen and PCL/Collagen/in situ HAPclay (CO60HC10) scaffolds. HUVECs wholly spread on the PCL/Collagen scaffolds by forming layers of tube-like cell networks. Similarly, PCL/Collagen/in situ HAPclay (CO60HC10) exhibited the formation of a vascular network. HUVECs did not adhere to the PCL scaffolds as much as the others. As shown in FIG. 8A, the cells did not form a tube-like network, resulting in reduced cell growth. In addition, scaffolds with and without sutures did not impact the growth or vascularization of the HUVECs. The corresponding pathway for collagen expression and YAZ and TAZ activation is demonstrated in FIG. 6C [FIG. 6C correct?].

In addition to the CD31 staining, we observed the expression of YAP and TAZ proteins. In FIG. 8B and FIG. 8C, YAP and TAZ were expressed more highly in cells on the PCL/Collagen/in situ HAPclay than in cells grown on the other scaffolds. PCL scaffolds showed less intensity of YAP and TAZ, consistent with CD31 expression (FIG. 8A). Furthermore, the sutures did not affect the growth and expression of YAP/TAZ proteins.

### Collagen Production on the CL60HC10 Scaffolds

The cells were seeded on a 3D scaffold composed of CO60HC10 after 23 days. In FIG. 13, panel (a), the red color indicates the presence of collagen expression, demonstrating that the hMSCs have differentiated and are producing extracellular matrix components like collagen. The blue signal shows the nuclei of the cells. This confirms cellular activity and collagen deposition within the scaffold. In contrast, FIG. 13, panel (b) represents a control with no collagen expression (green), as these scaffolds were not seeded with hMSCs. The lack of collagen implies no extracellular matrix deposition, which is consistent with the absence of cells in the scaffold. Both images use a 100 μm scale bar to give a sense of the microscopic size of the structures. The comparison highlights the role of hMSCs in promoting collagen expression when seeded on the scaffold.

### Tenocyte Differentiation Properties of PCL/In Situ HAPclay

TGFβ1 was employed to induce tenocyte cell differentiation from hMSCs cells in the 3D polymeric in situ HAPclay constructs of the disclosure. The transforming Growth Factor-β1 (TGFβ1) plays a pivotal role in various biological processes in the tendon. TGFβ signaling promotes tendon progenitor cell fate during early tendon development. TGFβ1 is involved in tendon repair by regulating tendon matrix production and organization. The activation of TGFβ signaling induces proliferation and inhibits apoptosis in tendon fibroblasts during the tendon healing process. Scleraxis (Scx) is an early-stage tenocyte and progenitor cell biomarker of the tendons. Tendon progenitors are marked by the expression of Scleraxis (Scx), a basic helix-loop-helix (bHLH) transcription factor critical for tenocyte differentiation. Cells at later stages of the lineage highly express type I collagen and TNMD.

As shown in FIG. 14, it was observed that PCL showed the lowest gene expression level (near 1-fold change), which is the control sample without any in situ HAPclay; whereas, 5% In situ HAPclay shows a moderate increase in SCX expression (panel (a)), with a significant upregulation compared to PCL. 10% In situ HAPclay demonstrated the highest significant SCX expression, with a substantial increase (around 4.5-fold change; ***, indicating highly significant upregulation with p<0.001).

The TNMD gene expression in PCL scaffolds is low (panel (b)), close to 1-fold, and serves as a control. In 5% In situ HAPclay PCL scaffolds, a moderate increase in TNMD expression was observed (around 2-fold change), still significant with p<0.01, indicating a significant upregulation compared to PCL as control. In contrast, in 10% in situ HAPclay PCL scaffolds shows the highest TNMD expression, signifying strong upregulation (with p<0.001).

RT-PCR data indicated that higher concentrations of in situ HAPclay promoted significantly stronger gene expression of both SCX and TNMD, essential markers for tenocyte cell differentiation. The strong upregulation under 10% in situ HAPclay suggests it is the most effective condition in enhancing gene expression in this experiment. Overall, in situ HAPclay affects tenocyte differentiation in the presence of growth factor, and the inclusion of more in situ HAPclay gives rise to significantly higher tenocyte differentiation. The CO60HC10 formulation of the repair scaffold contains 10% in situ HAPclay.

### Surgical Procedures

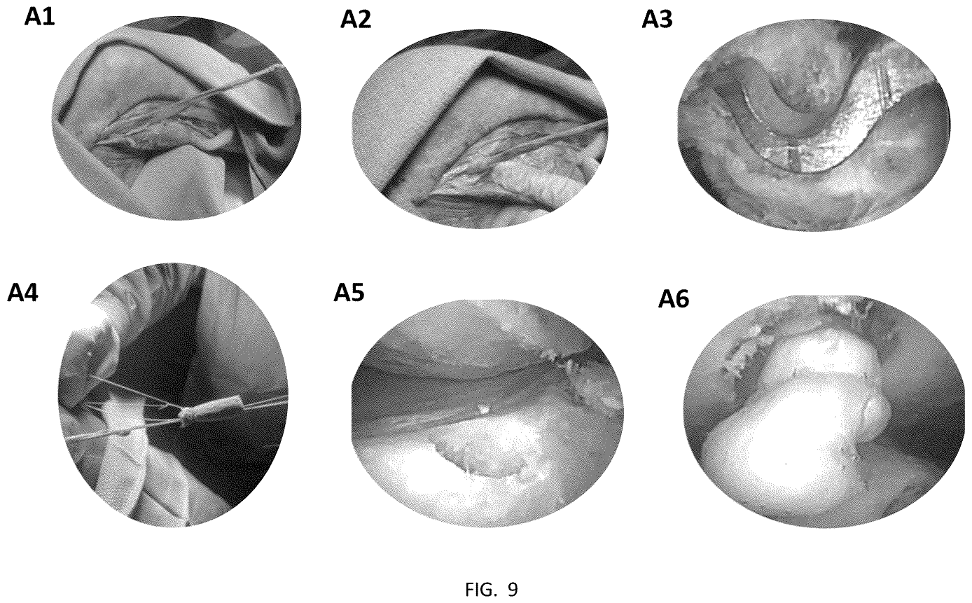

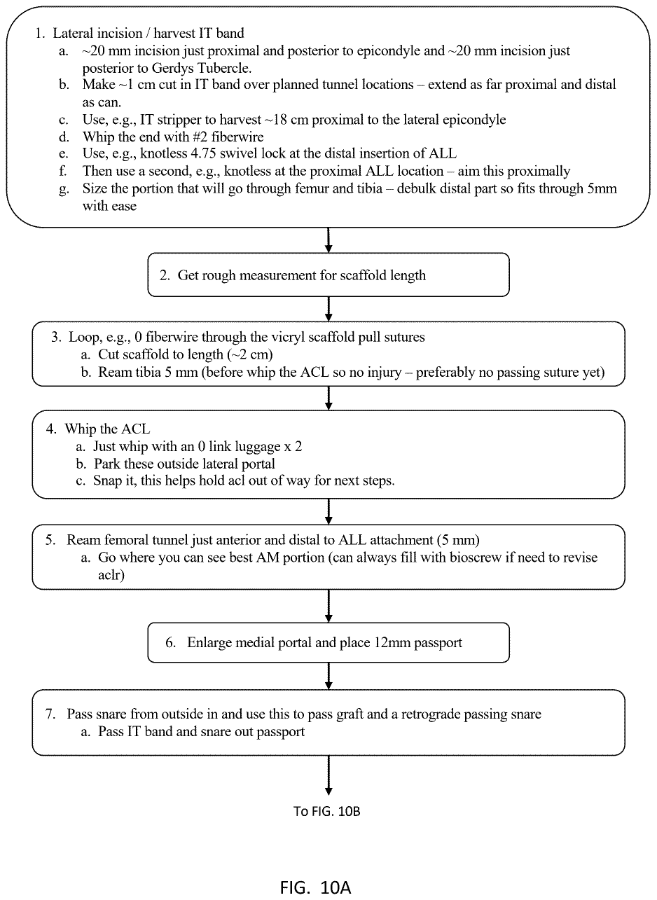

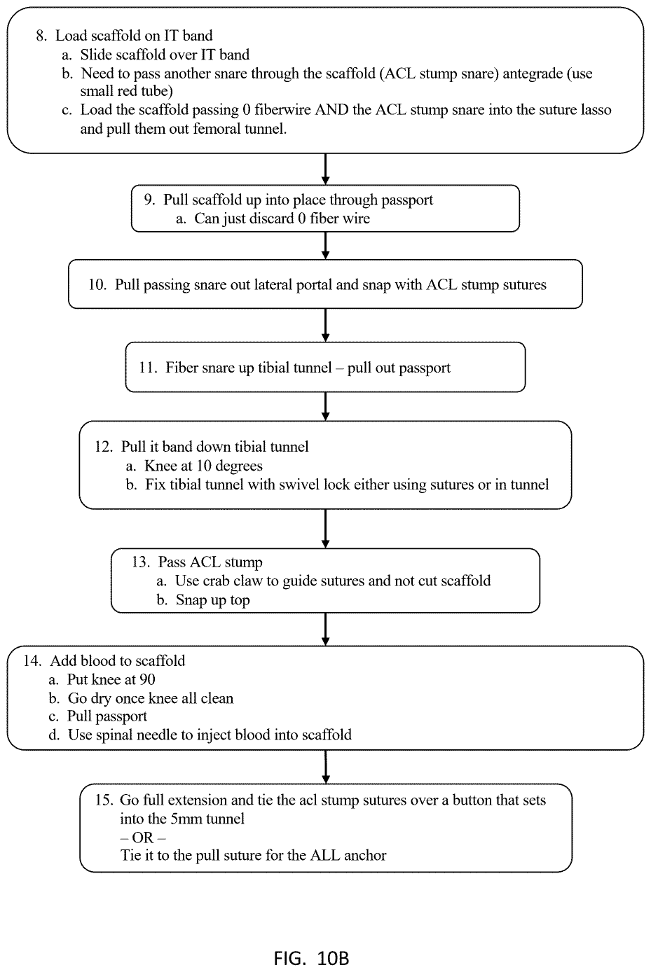

Procedure on cadaver model: the construct utilized for this described procedure (performed on a cadaver) consisted of a biological internal brace and a lateral tenodesis (both composed of a continuous 1 cm section of the iliotibial (IT) band and a CO60HC10 scaffold). In the surgery, the 1 cm strip of IT band remained attached distally (FIG. 9, panel A1), providing a biological pedicle, was routed proximally to create the lateral tenodesis (FIG. 9, panel A2), then entered the joint from outside in via a 5 mm tunnel (FIG. 9, panel A3). The CO60HC10 scaffold was then loaded over the top of the intra-articular portion of the IT band (Figure, panel 9A4), which then passed down through the tibial footprint and was fixated on the tibia, similar to a “mini” ACL reconstruction (Figure, panel 9A5). The sutures placed in the stump of the ACL were then pulled up the center of the scaffold, allowing an accurate “docking” of the ACL stump into the scaffold. The lateral tenodesis can be either a modified Lemaire or, by utilizing both distal and proximal fixation (FIG. 9, panel A6), an anterolateral ligament, as both techniques have been shown to provide similar protection to a maturing ACL.

### Surgical Outcomes

The use of scaffolds for ACL repair surgery is appealing; however, various challenges and complications associated with their design and performance often limit their application. A key component of a successful ACL repair scaffold is its high absorption rate, as well as both time-zero and progressively degrading mechanical properties. While prior scaffold studies have initially been met with great excitement, they have unfortunately demonstrated a suboptimal failure rate in young, active patient populations, which may be attributed to an early and aggressive return to activities. The proposed construct utilizes a biologically active, yet small, IT band within the CO60HC10 scaffold to address these limitations. From a mechanical properties perspective, previous studies used a synthetic “internal brace” consisting of four strands of Ethibond, with an ultimate strength of 268 N and a stiffness of 26 N (if balanced). This construct uses a small 1 cm strip of autologous IT Band, which has demonstrated an ultimate strength of 488 N and stiffness of 73 N/mm, providing more time-zero protection of the construct in both ultimate strength and significantly increased stiffness, as well as a source of biological activity and future graft integration. Incorporating the CO60HC10 scaffold significantly enhanced the mechanical properties, resulting in a Young's modulus of 2.2±0.63 MPa. This level of improvement in mechanical properties provided substantial support, contributing to the improved mechanical stability of the IT band/ACL repair construct. Furthermore, when the scaffold was strengthened with a zig-zag pattern of sutures, it substantially enhanced the tensile modulus up to 26.77±0.12 MPa. Moreover, the fabricated CO60HC10 scaffold diameter is 12 mm, allowing for placement in an over-the-top/extra physical routing of the IT band or all epiphyseal methods in skeletally immature patients.

Besides improved mechanical properties, the success of ACL repair surgery depends on the scaffold's ability to uptake the patient's blood and vascularization. The CO60HC10 scaffold demonstrated increased collagen type I protein expression and CD44 activation in hMSCs cells. The activation of CD44 triggers downstream signaling effects, leading to increased YAP and TAZ expression (FIG. 6C). This, in turn, leads to increased cell growth, differentiation, and vascular mimicry. YAP and TAZ are transcriptional co-activators, which, when activated, can induce an angiogenic response and promote vasculature. The immunostaining revealed elevated YAP/TAZ activity, along with CD31 surface marker staining, in the CO60HC10 scaffold, which reflects the capability of this scaffold to promote a significant intrinsic healing response. The enhanced YAP/TAZ/CD31 activity, along with the improved mechanical properties provided by the scaffolds (e.g., CO60HC10), makes it an ideal candidate for ACL repair. It also serves as a well-suited in vitro model system for elucidating key upstream and downstream signaling mechanisms (e.g., YAP/TAZ, Hippo) associated with specific biomechanical environments. The activation of YAP, TAZ, and CD31 expression in the CO60HC10 scaffold reflects the formation of vascular-like structures. Previous studies have demonstrated that the vascular ingrowth in the ACL originates from the synovial membrane, progressing downward and upward from the ligament stumps toward the mid-portion of the ligament. The CO60HC10 scaffold showed promising results in patient blood uptake (FIG. 2C), effectively preventing mixing with surrounding synovial fluids (Figure S5). This capability ensures localized blood retention, providing an optimal ACL regeneration and repair environment. Furthermore, the upregulation of collagen I when seeded with hMSCs indicates enhanced ECM remodeling and deposition. Collagen I is the primary structural component of ACL, and its increased presence in these scaffolds suggests that they provide a favorable microenvironment for mimicking native ACL tissue.

### A Detailed ACL Repair Surgical Method

A surgical method or procedure of the disclosure can include the following:

(1) Lateral incision/harvest IT band: An approximately 20 mm incision just proximal and posterior to epicondyle and 20 mm incision just posterior to Gerdys Tubercle are made. An approximately 1 cm cut in the IT band is made over the planned tunnel locations-extending as far proximal and distal as possible. An IT stripper is used to harvest 18 cm proximal to the lateral epicondyle. The end is whipped with #2 fiberwire. An anchor is used at the distal insertion of ALL. Then a second anchor is used at the proximal ALL location—aim this proximally. The portion is sized so that it will go through femur and tibia—debulk the distal part so it fits through 5 mm with ease.

(2) A rough measurement for scaffold length is acquired.

(3) 0 fiberwire is looped through the vicryl scaffold pull sutures, and the scaffold is cut to length (approximately 2 cm).

(4) The tibia is reamed 5 mm (before whip the ACL so no injury-no passing suture yet).

(5) The ACL is whipped. Whip with a luggage tag×2; park these outside lateral portal. Snap it, this helps hold ACL out of way for next steps.

(6) Ream femoral tunnel just anterior and distal to ALL attachment (5 mm). Go where you can see best AM portion (can always fill with bioscrew if need to revise aclr).

(7) Enlarge medial portal and place 12 mm flexible cannula.

(8) Pass snare from outside in and use this to pass graft and a retrograde passing snare. Pass IT band and snare out cannula.

(9) Load scaffold on IT band. Slide scaffold over IT band. Another snare is to be passed through the scaffold (ACL stump snare) antegrade (use small red tube). Load the scaffold passing 0 fiberwire AND the ACL stump snare into the suture lasso and pull them out femoral tunnel.

(10) Pull scaffold up into place through cannula. Can just discard 0 fiber wire.

(11) Pull passing snare out lateral portal and snap with ACL stump sutures.

(12) Fiber snare up tibial tunnel-pull out passport.

(13) Pull IT band down tibial tunnel, with knee at 10 degrees. Fix tibial tunnel with anchor or screw.

(14) Pass ACL stump. Use crab claw to guide sutures and not cut scaffold. Snap up top.

(15) Add blood to scaffold: Put knee at 90, go dry once knee is all clean; pull passport, and use spinal needle to inject blood into scaffold.

(16) Go full extension and tie the ACL stump sutures over a button that sets into the 5 mm tunnel—OR—tie IT to the pull suture for the anterolateral ligament (ALL) anchor.

### Materials and Methods

Certain materials used in the following methods are as follows. The polymer polycaprolactone (PCL) was obtained from Sigma-Aldrich. The amino acid 5-aminovaleric acid was obtained from Sigma-Aldrich. 1,4-dioxane was obtained from Sigma-Aldrich. Na₂HPO₄ and CaCl₂)) were obtained from J. T. Baker.

### Modification of MMT Clay

Sodium montmorillonite clay (Na-MMT clay; SWy-2) was purchased from the Clay Minerals Society (Wyoming). The received Na-MMT clay was modified with 5-aminovaleric acid as follows. Na-MMT was ground into fine powder and then screened through a no. 325 sieve (45 mm). About 5 g of this fine and sieved sodium MMT was placed in an oven for heating for 12 h at 60° C. The Na-MMT was then dispersed into 400 ml of deionized (DI) water pre-heated to 60° C. In another beaker, 1.9 g of amino acid (5-aminovaleric acid) was added to 100 ml of DI water pre-heated to 60° C. Further, the pH of the amino acid solution was maintained at 1.8 by addition of 0.1 N HCl. The amino acid solution was then added to the Na-MMT clay suspension, and the resulting solution was stirred vigorously for 1 h at a pH of 1.8 and at 60° C. The modified MMT was separated by centrifuging and further washed several times with DI water until the Cl-ions were removed completely from the clay. The filtrate obtained after centrifuging was titrated with 0.1 N silver nitrate. The titration procedure was repeated until no white precipitate was formed, which indicated the complete removal of chloride ions. Finally, the modified clay was placed in an oven for 24 h at 70° C. The clay was then ground and passed through a no. 325 sieve (45 mm).

### Preparation of In Situ HAPclay

Briefly, the in-situ HAPclay was prepared as follows: amino acid-modified Na-MMT clay was mixed into Na₂HPO₄ (JT Baker) solution and stirred for 2 hours. Thereafter, a 0.15 M CaCl₂) (JT Baker) solution was added to the clay mixture and stirred for another 9 hours while maintaining the pH at 7.5, which biomineralized the clay, growing hydroxyapatite inside clay galleries using the amino acids, thus making in situ, HAPclay. The precipitate was then separated by centrifuging followed by drying at 70° C., grinding, and sieving to obtain a fine in situ HAPclay powder.

### Preparation of PCL/Collagen In Situ HAPclay Scaffolds

The PCL (Sigma Aldrich) solution was prepared by dissolving 2 g of polymer in 40 ml of 1,4-dioxane (Sigma Aldrich) for stirring for 3 hours. In the next step, 1 g of the insoluble collagen type1 derived from the bovine tendon (Sigma Aldrich) was poured into the solution and stirred overnight at 60° C. Another solution was prepared by dispersing 0.4 g (10%) of in situ HAPclay in 20 ml of 1,4-dioxane and sonicating (20 minutes). The in-situ HAPclay solution was poured into polymer solution (PCL) and stirred for another 2.5 hours. Then, the prepared polymer HAPclay solution was poured into a tube, followed by freeze extraction to prepare PCL/Collagen/in situ HAPclay scaffolds. The same approach was used to fabricate the other scaffolds, and only the amount of collagen and in situ HAPclay was changed accordingly.

### Fabrication of Suture-Reinforced Composite Scaffolds Via Freeze-Drying Method

Braided absorbable sutures (Medtronic, USA) were integrated into scaffolds using a custom tubular mold system. A cylindrical tube served as the mold body, and a precision 3D-printed rod was positioned concentrically along its longitudinal axis. Sutures were passed parallel to the central rod and woven in predefined configurations, parallel, Zig-Zag, and combined patterns within the mold to create distinct reinforcement architectures. The scaffold matrix solution, composed of polycaprolactone (PCL), in situ HAPclay, and type I collagen, was then poured into the mold, fully embedding the woven sutures. The constructs were fabricated using the freeze-drying method, involving initial freezing at −20° C. for 12 h, producing porous scaffolds with uniformly integrated suture reinforcements and an interconnected pore network.

In other embodiments, a custom-designed and 3D-printed stem was placed in the tube before the solution was frozen to create a cylindrical hole through the middle of the scaffold. In addition, a custom designed device was 3D printed to allow for stretching degradable sutures through the walls of the scaffold and also create a cylindrical home through the middle of the scaffold. The same approach was used for fabricating the other scaffolds, and only the amount for the in situ HAPclay has been changed accordingly.

### Preparation of Synovial Fluids

To assess the degradability profile and its influence on mechanical properties, PCL/Collagen/in-situ HAPclay scaffolds with the mentioned geometry were fabricated. In the first step, the scaffolds were immersed in the solution of the synovial fluid containing hyaluronic acid, immunoglobulin, and Ringer's solution for 29 days. The degradation characteristics of newly developed scaffolds were examined through loss of mass, alterations in mechanical behavior, and scaffold morphological change upon exposure to the synovial fluid. The biodegradation procedure was carried out in accordance with the ASTM D5338-15 standard. The synovial fluid allowed for degradation testing at Day 0 (Control), Day 7, Day 14, Day 21, and Day 30, closely mimicking the natural environment for the degradation period. Every scaffold was placed inside a tube, sealed, and then placed into an incubator at 37° C. The weight of the scaffolds was measured before and after drying. The weight loss percentage, as one of the criteria for the degradation investigation, was calculated based on the following equation:

$$Weight loss \% = \frac{{W}_{o} - {W}_{f}}{{W}_{o}} \times 100 \qquad ( 1 )$$

where W₀ is the initial weight, and W_{f} is the degraded weight of the scaffolds. Before taking measurements, the samples were gently dried with filter paper to remove any surface moisture.

### PBS Absorption of the Scaffolds

The in vitro bioactivity assessment of the scaffolds was conducted by immersion in PBS (phosphate-buffered saline) for different durations: 2 minutes, 5 minutes, 15 minutes, 1 hour, and 24 hours. The choice of these specific time frames reflected the estimated time required for the scaffolds to absorb blood effectively before the ACL IT-band surgery. The scaffolds were initially shaped into cylinders measuring 4 mm long and 12 mm in diameter, with an internal hole of 6 mm, consistent with dimensions appropriate for ACL surgery. Subsequently, the scaffolds were dried in a fume hood for 2 hours. The measurements were conducted a minimum of five times, and the results were averaged. The percentage of water absorption was then calculated using Eq. (2):

$$Absorption Factor = \frac{Wt - W 0}{W 0} \times 100 \qquad ( 2 )$$

To ensure the accuracy and precision of the scaffolds' measurements, the samples were removed from PBS (phosphate-buffered saline) before weighing and dabbed with filter paper to eliminate surface moisture.

### Absorption Performance of Scaffold in Cadaveric Model

The surgical technique utilizes a continuous (approximately) 1 cm segment of the iliotibial (IT) band, which remains attached distally to maintain a biological pedicle. This segment is combined with a scaffold (e.g., CO60HC10) to create a construct that serves both as a biological internal brace and as a lateral tenodesis. The IT band is guided proximally to perform the lateral tenodesis and then introduced into the joint through a 5 mm tunnel. The CO60HC10 scaffold is positioned around the intra-articular portion of the IT band, which is subsequently routed through the tibial footprint and fixed onto the tibia. The ACL stump is carefully sutured and drawn into the scaffold to ensure accurate placement. Following scaffold implantation within the knee, blood is injected into the scaffold to promote integration.

### Mechanical Properties

The compression and tensile experiments were performed utilizing the EZ-X Series Universal Electromechanical Test Frames (Shimadzu, model EZ-LX HS). Following the guidelines of ASTM D638, a consistent deformation speed of 5 mm/min was employed in each test for all specimens, allowing up to 10% strain. Five samples were utilized for each data point, and the associated load-displacement information was measured. The Young's modulus of the scaffold samples was calculated by analyzing the slope of the initial linear portion of the stress-strain curve. The response of the newly developed scaffolds comprised of PCL, Collagen Type 1, and in-situ HAPclay, in response to the compressive loading before and after immersion in synovial fluids, on Day 0 (Control), Day 1, Day 7, Day 15, Day 22, and Day 29, has been investigated. The tensile test for measuring the mechanical properties of sutures was also performed utilizing the EZ-X Series Universal Electromechanical Test Frames (Shimadzu, model EZ-LX HS), with a consistent deformation speed of 5 mm/min.

### Scanning Electron Microscopy (SEM)

The surface morphology of the PCL/Collagen Scaffolds was examined before and after degradation using SEM (Scanning Electron Microscope) operated at an acceleration voltage of 20 kV (SU8010, Hitachi, Japan). Before the analysis, the film surfaces were coated with a layer of gold.

### Differential Scanning Calorimetry (DSC)

The scaffolds were analyzed using a Discovery DSC 2500 differential scanning calorimeter for DSC measurements. The specimens (5-10 mg) were subject to a heat-cool-heat cycle (0 to 80° C.) with a heating/cooling rate of 10° C. min-1. Nitrogen was used as a purge gas. The glass transition temperature values were determined on these second heating cycles.

### Fourier Transform Infrared Spectroscopy (FTIR)

FTIR experiments were performed utilizing a ThermoNicolet NEXUS™ 870 FTIR spectrometer equipped with a film beam splitter. Samples were positioned using film windows and subsequently secured in the universal sample holder for conducting the experiments. Spectra were acquired in transmission mode at a resolution of 4 cm⁻¹, with a mirror velocity set at 0.169 cm/s.

### X-Ray Diffraction (XRD)

XRD data for powdered clay samples were collected using a Rigaku X-ray diffractometer. The d-spacing of the scaffold was calculated from the XRD data according to Bragg's diffraction law. The test was conducted over a 20 range of 15-50° at a rate of 2° min-1. Before the XRD scan, film of PCL, PCL/Collagen, and PCL/Collagen/in situ HAPclay modified with amino valeric acid samples were placed in an aluminum holder.

### Cell Culture

Human mesenchymal stem cells (hMSCs) were cultured using an MSCGM bullet kit medium (Lonza, USA). Human umbilical vein endothelial cells (HUVECs) were cultured using endothelial cell growth kit-BBE (ATCC, USA). All cultures were maintained at 37° C. and 5% CO₂ in a humidified incubator. Prior to seeding, all scaffolds were first sterilized under UV light for 45 minutes and then immersed in 70% ethanol for 12 hours. Next, they were washed in PBS three times for 12-hour intervals and immersed in the culture medium. Lastly, scaffolds were seeded with 1×10⁵ hMSCs per scaffold and kept for 4 hours before adding culture medium. Cell-seeded scaffolds were cultured for 1, 3, 7, and 10 days. HUVECs were seeded on 10-day hMSC-seeded scaffolds for 5 hours.

### Growth Factor Preparation and its Concentration

TGF-beta (10 g) was purchased from Promega in powder form. Solutions were prepared following the manufacturer's protocol at 1 μg/ml concentration. Sterilized scaffolds were immersed in the freshly made TGF-beta solution for 24 hours. After 24 hours, TGF-beta-coated samples were kept in hMSC complete media (Lonza: PT-3001) for at least 24 hours.

### For Gene Expression

Total RNA was extracted from the cell-seeded scaffolds using the Direct-zol RNA MiniPrep kit from Zymo Research. The concentration of the isolated RNA was determined using the Nanodrop ND 2000 from Nanodrop products. Next, cDNA synthesis was performed using 2 μg/μl of RNA, random primers, and M-MLV reverse transcriptase from Promega in a PCR thermal cycler from Applied Biosystems. The real-time polymerase chain reaction (PCR) was conducted using the 7500 Fast Real-Time PCR system from Applied Biosystems. A final volume of 20 μl was prepared by adding forward primer, reverse primer, SYBR Green dye, and cDNA. The thermal profile consisted of a holding stage (2 minutes at 50° C. and 10 minutes at 95° C.) followed by a cycling stage (40 cycles of 15 seconds at 95° C. and 1 minute at 60° C.). The mRNA expressions of genes, including scleraxis (Scx) and Tenomodulin (TNMD) were measured and compared to the expression of the housekeeping gene glyceraldehyde-3-phosphate-dehydrogenase (GAPDH) for normalization. The analysis of target gene expressions was performed using the comparative Ct method.

### Live Dead Assay

Live/dead assay was performed to evaluate the viability of hMSCs on the different scaffolds, over time. Samples were imaged at 1, 3, 7, and 10 days. According to manufacturer instructions, cells were stained with a Live/Dead™ Cell Imaging Kit (Thermofisher Scientific, Germany) according to the manufacturer's protocol. Briefly, the solution was prepared by mixing the Calcein AM solution and BOBO-3 iodide and diluting it to a working concentration with PBS. Lastly, samples were incubated in the live/dead solution for 25 minutes at room temperature. Samples were imaged using a JPK Nanowizard Bio-AFM confocal system and FITC/TRITC filters.

### Immunofluorescence

Seeded scaffolds (7-day hMSCs, 10-day hMSCs, and HUVEC/10-day hMSCs) were fixed with 4% paraformaldehyde for 45 minutes. Following, samples were washed with PBS three times, 5-minute interval. Permeabilization with 0.2% Triton-x was done to all the samples and then washed with PBS three times, 5-minute intervals. Next, the samples were blocked with 1× blocking buffer (Abcam) for 50 min, followed by incubation with the primary antibody overnight at 4° C. Nestin (Cell signaling), Collagen-1 (Abcam), CD44 (Abcam), CD31 (Abcam), YAP (cell signaling), and TAZ (cell signaling) antibodies were diluted in a blocking buffer. Finally, Alexa Flour 488/568/CY5 conjugated secondary antibodies corresponding specifically to the origin of the used primary antibodies were added at 1:250 dilution and incubated for 45 min at room temperature. The nuclei were counterstained with DAPI. The samples were imaged under confocal microscope (JPK Nanowizard Bio-AFM).

### Statistical Analysis

Statistical analyses were performed using GraphPad Prism version 10.04. Data are presented as mean±standard deviation (SD). One-way or two-way ANOVA tests were utilized to analyze the data, with Tukey's post hoc test applied for multiple comparisons. Differences were considered statistically significant at p-values below 0.05.

### CONCLUSION

The novel PCL/Collagen/in situ HAPclay scaffolds described herein (e.g., CO60HC10) with degradable sutures and a cylindrical hole in the center of the scaffold to place the Iliotibial (IT) band and repaired torn ACL have great potential for improving ACL repair. The scaffold absorption study described above demonstrated good blood absorption during the surgery, effectively retaining the absorbed blood throughout the procedure, highlighting its ability to support the biological environment necessary for ACL healing. Additionally, the scaffold demonstrated adequate degradation rates, improved collagen production, and vasculature formation, which are critical for long-term ACL regeneration and function. The slower degradation rates of these scaffolds, as compared to the existing collagen scaffolds, allow the scaffold to aid in ACL regeneration and healing over a longer period. The cylindrical hole in the center of the scaffold allows for the placement of the IT band or repaired ACL such that the ligament is covered by the bioactive scaffold, creating an intimate contact between the two. This allows for better integration of the IT band and the cells and tissues growing in the scaffold. During the ACL repair, tunnels are drilled through the bone in the knee, and the scaffold and the IT band are pulled through the tunnel. The degradable suture reinforcement of the scaffolds maintains the integrity of the scaffold during surgery and is used to pull the scaffold through the tunnel(s). Its tensile properties have increased substantially by reinforcing the (e.g., CO60HC10) scaffold with a parallel or zig-zag pattern of sutures. The enhanced mechanical properties and reduced size allow for the repair via minimally invasive surgery (MIS) or laparoscopy, reducing the patient's time for recovery. Successful ACL IT band surgery with the new scaffold in a cadaver model confirmed feasibility.

1. A composition comprising:
a composite comprising;
a biocompatible clay;
a polymer;
a polypeptide; and
a calcium-based mineral;
a bioabsorbable reinforcement structure;
wherein the composite forms a three-dimensional scaffold with a hole formed therethrough along an axis of the scaffold; and
the bioabsorbable reinforcement structure is located on an outer surface of the scaffold, an inner surface of the scaffold, within the scaffold, or combinations thereof.

2. The composition of claim 1, wherein the biocompatible clay comprises bentonite, beidellite, hectorite, Kelocyte, montmorillonite, nontronite, saponite, or combinations and other silicate minerals thereof.

3. The composition of claim 1, wherein the biocompatible clay is modified with an amino acid selected from amino valeric acid, glycine, alanine, valine, leucine, isoleucine, proline, phenylalanine, tryptophan, methionine, serine, threonine, cysteine, tyrosine, asparagine, glutamine, aspartic acid, glutamic acid, lysine, arginine, histidine, or combinations thereof.

4. The composition of claim 1, wherein the polymer comprises albumin, alginate, cellulose, chitin, chitosan, collagen, gelatin type A, gelatin Type B, heparin, regenerated silk polymer, polysaccharide, poly(amino acid), polyanhydride, polyester, poly(alpha-hydroxy acid), poly(lactone), poly(orthocarbonate), poly(orthoester), poly(phosphoester), polyphosphazenes, polyacrylonitrile, polycaprolactone, poly(delta-valerolactone), poly(1,5-dioxepan-2-one), poly(epsilon-aprolactone), poly(ester urethane), polygalactouronic acid, poly(gamma-butyrolactone), polygly colic acid, poly(alpha-hydroxy acids), polyhydroxyalkanoate, polyhydroxybutyric acid, poly(3-hydroxybutyrate-co-3-hydroxyvalerate, polyimide, polylactic acid, poly(lactic-co-gly colic acid), poly(lactic acid-co-caprolactone), poly(trimethylene carbonate), poly-8-valerolactone, or blends, combinations, and mixtures thereof.

5. The composition of claim 4, wherein the polymer is polycaprolactone (PCL).

6. The composition of claim 1, wherein the polypeptide comprises collagen, a modified collagen, a collagen-related peptide, or a collagen-mimetic peptide or combinations thereof.

7. The composition of claim 6, wherein the collagen is collagen type I.

8. The composition of claim 1, wherein the calcium-based mineral comprises a bioceramic, the bioceramic comprising a mono-, di-, tri-, [alpha]-tri-, [beta]-tri-, tetra-calcium phosphate, hydroxyapatite, a calcium sulfate, a calcium oxide, a calcium carbonate, a magnesium calcium phosphate, or combinations thereof.

9. The composition of claim 1, wherein the calcium-based mineral is hydroxyapatite in the form of in situ mineralized hydroxyapatite formed on the biocompatible clay.

10. The composition of claim 1, wherein the bioabsorbable reinforcement structure comprises a monofilament thread, polyfilament thread, mesh, braided suture, or combinations thereof.

11. The composition of claim 1, wherein the clay comprises between about 5 wt. % to about 12 wt. % of the composite.

12. The composition of claim 1, wherein the polymer and polypeptide together comprise between about 10 wt. % to about 90 wt. % of the composite.

13. The composition of claim 1, wherein the composition further comprises an additional agent of an amino acid, anesthetic, antibiotic, antiangiogenic agent, antibody, anticoagulant, biomaterial, bone morphogenetic proteins, carbohydrate, cell, drug, electrolyte, growth factor, immunomodulator, inorganic material, lipid, mineral, oligonucleotide, osteoblast, osteoclast, osteo stem cell, peptide, progenitor, protein, therapeutic agent, tissue, tissue or cell aggregate, vasoactive agent, and combinations thereof,
wherein the additional agent is between 0.01 wt. % and 50 wt. % of the composition.

14. The composition of claim 13, wherein one or more of the additional agent is released by a controlled release and/or sustained release.

15. A method for preparing the composition of claim 1 comprising:
dissolving the clay in an aqueous solvent to form a dissolved clay;
adding a calcium-based mineral to the dissolved clay to form a mineralized clay precipitate;
separating, drying and grinding the mineralized clay precipitate to form a mineralized clay powder;
dissolving the polymer in a solvent to form a dissolved polymer;
adding the polypeptide to the dissolved polymer to form a polymer and polypeptide mixture;
adding the mineralized clay powder to the polymer and polypeptide mixture to form a clay, polymer, and polypeptide mixture;
adding a bioabsorbable reinforcement structure within a mold of a desired three dimensional shape;
adding the clay, polymer, and polypeptide mixture to the mold;
placing an occluding device or stem through the clay, polymer, and polypeptide mixture to form a hole in the composite once the mixture is in solid form and the occluding device or stem is removed;
freeze extracting the clay, polymer, and polypeptide mixture; and
forming a scaffold in the desired three dimensional shape with a hole therethrough and a bioabsorbable reinforcement structure, once removed from the mold and removing the stem.

16. The method of claim 15, wherein the clay is modified with an amino acid prior to dissolving in the aqueous solvent.

17. The method of claim 15, wherein the scaffold has a length of about 4 mm, an outer diameter of about 12 mm, and an inner hole diameter of about 6 mm.

18. The method of claim 15, further comprising seeding the scaffold with cells;
wherein the cells comprise one or more of (1) human mesenchymal stem cells (hMSCs) alone, or human umbilical vein endothelial cells (HUVECs) on scaffolds previously seeded with hMSCs.

19. A method for repairing a tear or rupture of a ligament or tendon, comprising:
(i) obtaining a graft;
(ii) sliding a composition of claim 1 over the graft;
(iii) anchoring one or more ends of the graft to repair or replace the torn or ruptured ligament or tendon.

20. The method of claim 19, wherein the graft is a strip of IT band or synthetic internal brace.

21. A kit for ligament or tendon repair comprising:
a composition according to claim 1; and
instructions for use in a surgical procedure for repairing or replacing a torn or ruptured ligament or tendon.

22. The kit of claim 21, further comprising surgical instruments for harvesting a graft, creating bone tunnels, and positioning the composition over the graft.

23. A composition according to claim 1, wherein the composition comprises:
polycaprolactone at about 40-70% by volume;
collagen type I at about 40-70% by volume;
in situ hydroxyapatite-clay at about 5-12% by volume;
wherein the clay is montmorillonite modified with amino valeric acid; and
wherein a bioabsorbable suture is oriented in a zig-zag pattern within the scaffold.

24. The composition of claim 23, wherein the composition comprises about 60% polycaprolactone, about 30% collagen type I, and about 10% in situ hydroxyapatite-clay by volume.
